(19) United States
(12) Patent Application Publication
(10) Pub. No.: US 2006/0168668 A1
(43) Pub. Date: Jul. 27, 2006

(54) Phosphodiesterase 2a and methods of use

(75) Inventors: Daguang Wang, Montvale, NJ (US); Bozena Bugaj-Gaweda, Westwood, NJ (US)

Correspondence Address: DARBY & DARBY P.C., P. O. BOX 5257, NEW YORK, NY, 10150-5257, US

(73) Assignee: Memory Pharmaceuticals Corporation, Montvale, NJ (US)

(21) Appl. No.: 10/545,501

(22) PCT Filed: Apr. 1, 2004
(86) PCT No.: PCT/US04/10060

§ 371 Date: Aug. 12, 2005

Related U.S. Application Data
(60) Provisional application No. 60/459,977, filed on Apr. 4, 2003.

(51) Int. Cl. A01K 67/00; C12Q 1/68; G06F 19/00; C07H 21/04; C12P 21/06; C12N 9/16; G01N 33/50

(52) U.S. Cl. 800/8; 435/6; 435/69.1; 435/196; 435/320.1; 435/325; 530/388.26; 536/23.2; 702/20

(57) ABSTRACT

The invention relates to phosphodiesterase PDE2A isoforms, as well as nucleic acids encoding such polypeptides. The invention further relates to methods for utilizing such polypeptides in diagnostic assays and in methods of screenin potential modulators, especially inhibitors, of the novel PDE2A disclosed herein. The polypeptides of the invention are involved in many physiological processes including, e.g. the formation of memory, cognitive function, and other disease and conditions which involve or are mediated by PDE2A and its signaling pathways.

This application claims the benefit of U.S. Provisional Application Ser. No. 60/459,977, filed Apr. 4, 2003, which is hereby incorporated by reference in its entirety.

### BACKGROUND OF THE INVENTION

The phosphodiesterases (PDEs) represent a family of enzymes that catalyze the hydrolysis, of various cyclic nucleoside monophosphates (including cGMP and cAMP). These cyclic nucleotides act as second messengers within cells, and as messengers, carry impulses from cell surface receptors having bound various hormones and neurotransmitters. PDEs act to regulate the level of cyclic nucleotides within cells and maintain cyclic nucleotide homeostasis by degrading such cyclic mononucleotides resulting in termination of their messenger role.

Eleven PDE gene families (PDE1-PDE11) have been identified so far, based on their distinct amino acid sequences, catalytic and regulatory characteristics, and sensitivity to small molecule inhibitors. PDE2 gene family members contain two tandem GAF domains (GAFa and GAFb) and hydrolyze both cAMP and cGMP. GAF domains in PDEs are regulatory modules localized upstream of the catalytic site. The crystal structure of mouse PDE2 regulatory segment, which contains GAFa and GAFb, has recently been solved. Although very similar in structure, GAFa and GAFb serve distinct roles. GAFa is part of the dimerization domain, and GAFb binds cGMP to allosterically regulate PDE2 enzyme activity.

PDE2 is highly expressed in the brain (Sonnenburg et al., J Biol Chem, 266:17655-61 (1991); Rosman et al., Gene, 191:89-95 (1997)). PDE2 has been shown to couple to NMDA receptor to regulate cGMP level upon NMDA stimulation in primary cortical and hippocampal neurons (Suvarna, J Pharmacol Exp Ther, 302:249-56 (2002)). NO/cGMP signaling pathway in CNS has been subjected to extensive study and has been shown to be important for many synaptic plasticity events, such as LTP (Hawkins et al., J Neurobiol, 25:652-65 (1994); Jaffrey, Annu Rev Cell Dev Biol, 11:417-40 (1995)). NO has been proposed to act as a retrograde messenger, synthesized at the postsynaptic terminal by a Ca++-dependent NOS, diffusing back to the presynaptic terminal to trigger long-lasting increased neurotransmitter release. NO activates soluble guanylyl cyclase to synthesize cGMP, which in turn is regulated by a cGMP-degrading DE, most likely PDE2.

Three PDE2A isoforms have been cloned from three different species, PDE2A1 from bovine (Trong et al., Biochemistry, 29:10280-8 (1990); Sonnenburg et al., J Biol Chem, 266:17655-61 (1991)), PDE2A2 from rat (Yang et al., Biochem Biophys Res Commun, 205:1850-8 (1994)) and PDE2A3 from human (Rosman et al., Gene, 191:89-95 (1997)). Sequence alignment reveals that the three isoforms are highly homologous except at the extreme N-terminal, suggesting alternative N-terminal exon usage during splicing. The function of the unique N-terminal exons has not been determined, although it is suggested that they could be responsible for particulate or soluble localization of the enzyme, as seen with other PDEs (for review see Houslay et al., Adv Pharmacol, 44:225-342 (1998); Francis et al., Progress in Nucleic Acid Research and Molecular Biology, 65:1-52 (2001); Houslay, Biochem J, (2002)).

### BRIEF DESCRIPTION OF THE DRAWINGS

FIGS. 1A-1B. Nucleotide sequence of cDNA encoding human PDE2A4, hsPDE2A4 (SEQ ID NO: 1). The initiation, downstream stop, and in-frame upstream stop codons are bolded and italicized.

FIG. 2. Translated amino acid sequence of human PDE2A4 isoform (SEQ ID NO: 2).

FIG. 3. EHNA inhibition of hsPDE2A4 activity. QM7 lysate containing recombinant human PDE2A4 was used in inhibition assay with increased concentration of inhibitors (EHNA and Rolipram; 10⁻⁹-10⁻⁴ μM). The data and graph were analyzed and generated using Prism software.

FIG. 4. cGMP stimulation of hsPDE2A4 activity. QM7 lysate containing recombinant human PDE2A4 was used in stimulation assay. The cAMP substrate concentration was 30 μM ([³H]-cAMP/cAMP). The enzyme activities in the absence or presence of 10 μM cGMP were compared. Data analysis was performed using Prism software.

FIG. 5. Tissue expression pattern for human PDE2A1, PDE2A3, and PDE2A4 isoforms. A 5′ primer unique to each isoform and a common 3′ primer were used to PCR human Multiple Tissue cDNA panel from Clontech, according to recommended protocol from manufacturer. The PCR reactions were allowed to proceed for 35 cycles, then equal volume of PCR products from each tissue were analyzed on 2% TBE agarose gel. PCR of GAPDH was performed at the same time to normalize the tissue panel and gel loading.

FIG. 6. Nucleotide sequence of cDNA encoding human PDE2A2, hsPDE2A2 (SEQ ID NO: 13). The initiation and downstream stop codons are bolded and italicized.

FIG. 7. Translated amino acid sequence of human PDE2A2 isoform (SEQ ID NO: 14).

FIG. 8. Amino acid comparison of PDE2 isoforms. bvPDE2A1 (bovine) (SEQ ID NO: 17); hsPDE2A1 (human) (SEQ ID NO: 18); hsPDE2A2 (human) (SEQ ID NO: 14); rnPDE2A2 (rat) (SEQ ID NO: 19); hsPDE2A3 (human (SEQ ID NO: 20); hsPDE2A4 (human) (SEQ ID NO: 2).

### DESCRIPTION OF THE INVENTION

The present invention relates to all facets of novel polynucleotides, the polypeptides they encode, antibodies and specific binding partners thereto, and their applications to research, diagnosis, drug discovery, therapy, clinical medicine, forensic science and medicine, etc. In particular, the present invention is directed to all aspects of polynucleotides and polypeptides that are members of a class of related isoforms belonging to the “A” subtype of the cAMP (cyclic adenosine 5′ monophosphate) and cGMP (cyclic guanosine 5′ monophosphate) phosphodiesterase 2 (PDE2) family of enzymes. The invention also relates to fragments and variants of the polypeptide and polynucleotides, and methods of use thereof. The polypeptides are involved in many physiological processes including, e.g., the formation of memory, neurological disorders, cancer, etc., and relate to any diseases, disorders, and conditions associated and/or mediated by the signaling pathways comprising a PDE of the present invention.

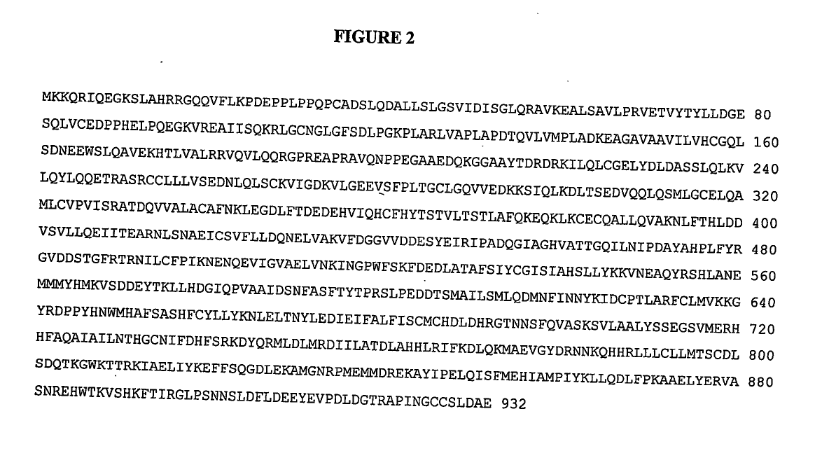

The present invention provides a mammalian PDE2A4 isoform, including a full-length human PDE2A4 polypeptide, e.g., as represented by SEQ ID NO: 2 as shown in FIG. 2. The polypeptide represented by SEQ ID NO: 2 has 932 amino acids with a calculated molecular weight of 104912 Daltons. Located in the N-terminal portion of the polypeptide of the invention is a novel 14-mer fragment (an unbroken sequence of 14 amino acids, i.e. an uninterrupted stretch of 14 consecutive amino acids), which is unique to PDE2A4 and represented by SEQ ID NO: 4. This sequence is sometimes referred to herein as the unique 14-mer, or generically as the 14-mer. The sequence of the 14-mer is MKKQRIQEGKSLAH (SEQ ID NO: 4).

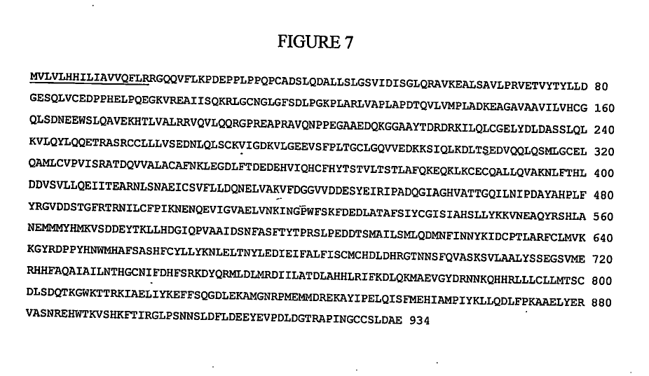

The present invention also provides a human PDE2A2 isoform, including a full-length PDE2A polypeptide, e.g., as represented by SEQ ID NO: 14 as shown in FIG. 7. The polypeptide represented by SEQ ID NO: 14 has 934 amino acids with a calculated molecular weight of 105103.86 dalltons. Located in the N-terminal portion of the polypeptide of the invention is a novel 17-mer fragment (an unbroken sequence of 17 amino acids, i.e. an uninterrupted stretch of 17 consecutive amino acids), which is unique to PDE2A2 and represented by SEQ ID NO: 16. This sequence is sometimes referred to herein as the unique 7-mer, or generically as the 17-mer.

The sequence of the 17-mer is MVLVLHHLIAVVQFLR (SEQ ID NO: 16).

The invention also relates to an isolated polypeptide comprising the N-terminally-located sequences of the polypeptide, especially SEQ ID NO: 4 or 16, or comprising a fragment or variant of SEQ ID NO: 4 or 16.

Polypeptides of the present invention can possess one or more of the following biological activities or properties, including, e.g., but not limited to, immunological activity (e.g., capable of eliciting an immune response and/or generating antibodies), phosphodiesterase activity, ability to hydrolyze nucleoside monophosphates (including cAMP and cGMP), having a lower Michaelis constant for cGMP than for cAMP, sensitive to EHNA (erythro-9-(2-hydroxy-3-nonyl) adenine hydrochloride), ability to bind to cGMP and related substrates, ability to bind to cAMP and related substrates, etc

The aforementioned activities can be determined routinely. For example, phosphodiesterase activity (including the ability to hydrolyze cAMP and/or cGMP) can be measured as described in the examples below. In addition, the activity can be determined as described in, e.g., U.S. Pat. No. 6,500,610 or 6,569,638; Thompson et al., Methods Enzymol, 38: 205-212, 1974; Hansen and Beavo, Proc. Natl. Acad. Sci., 79: 2788-2792, 1982; Rosman et al., Gene, 191:89-95, 1997. Binding assays for cAMP and cGMP can be performed routinely, e.g., as described in U.S. Pat. No. 6,569,638 and Francis et al., J. Biol. Chem., 255:620-626, 1980.

Another aspect of the invention is an isolated cDNA, which encodes a full-length PDE2A4 protein. A typical cDNA is represented by SEQ ID NO: 1. The cDNA has been cloned and deposited as plasmid HSPDE2A4 on Jan. 29, 2003, with the American Type Culture Collection (ATCC), 10801 University Blvd., Manassas, Va. 20110-2209, U.S.A. under the provisions of the Budapest Treaty for the International Recognition of the Deposit of Microorganisms for the Purposes of Patent Protection and was accorded ATCC Patent Deposit Designation No. PTA4986. Located near the 5′ end of the cDNA is the sequence of SEQ ID NO: 3, which encodes the 14-mer polypeptide discussed above. The cDNA sequence is:

(SEQ ID NO: 3)

5′-ATGAAGAAACAGAGGATCCAGGAGGGGAAATCGCTTGCCCAC-3′

Another aspect of the invention is an isolated cDNA, which encodes a full-length PDE2A2 protein. A typical cDNA is represented by SEQ ID NO: 13. Located near the 5′ end of the cDNA is the sequence of SEQ ID NO: 15, which encodes the 17-mer polypeptide discussed above. The cDNA sequence is

(SEQ ID NO: 15)

5′-ATGGTCCTGGTGCTGCACCACATCCTCATCGCTGTTGTCCAATTCCT
CAGG-3′

Thus, the invention relates, e.g., to an isolated polynucleotide comprising the cDNA sequence of SEQ ID NO: 1, 13, or fragments or variants thereof. The invention also relates to an isolated polynucleotide comprising the 5′-terminally located sequence of the cDNA, SEQ ID NO: 3, 15, a fragment or variant thereof, or complements thereof. For example, the invention encompasses oligonucleotides within polynucleotides comprising the nucleic acid sequence of SEQ ID NO: 1, 3, 13, and 15.

Another aspect of the invention is an isolated polynucleotide which comprises a nucleotide sequence that codes without interruption for the polypeptide of SEQ ID NO: 2, 4, 14, 16, or a fragment or variant of SEQ ID NO: 2, 4, 14, 16, or that is the complement of a sequence that codes without interruption for the polypeptide of SEQ ID NO: 2, 4, 14, 16 or a fragment or variant thereof A polynucleotide, which “codes without interruption” refers to a polynucleotide having a continuous open reading frame (“ORF”) as compared to an ORF, which is interrupted by introns or other noncoding sequences.

The invention also relates to methods of making the above-described polypeptides or polynucleotides, e.g., methods of making constructs which comprise and/or express the polynucleotide sequences; methods of transforming cells with constructs capable of expressing the polypeptides, culturing the transformed cells under conditions effective to express the polypeptides, and harvesting (recovering) the polypeptides); to antibodies, antigen-specific fragments, or other specific binding partners which are specific (selective) for the polypeptides; to methods of detecting a disease or condition, or a susceptibility to a disease or condition that is associated with aberrant expression (e.g., under- or over-expression) of the polypeptides or polynucleotides, or with variant forms (e.g., mutants, polymorphisms, SNPs, etc.) of the polypeptides or polynucleotides; to methods of treating a disease conditions associated with, or mediated by, PDE or the signaling pathways comprising it; methods of treating, preventing, or modulating any of the below mentioned conditions or diseases, e.g., treating memory dysfunctions, stimulating memory formation; treating cognitive dysfunction; to methods of using polypeptides, polynucleotides or antibodies of the invention to detect the presence or absence, and/or to quantitate the amounts, of the polypeptides and polynucleotides of the invention in a sample; to methods of detecting mutations in the polypeptide or polynucleotide sequences which are associated with a disease condition; to methods of using the polypeptides or polynucleotides, or cells transformed with the polynucleotides, to screen for potential therapeutic agents, e.g., agents which modulate the activity or amounts of the polynucleotides or polypeptides; to transgenic animals which express the polypeptides or knockout animals which do not express the polypeptides; or for other potential uses.

For example, the invention relates to an isolated polypeptide comprising the amino acid sequence of SEQ ID NO: 2, 4, 14, 16, or a fragment or variant of SEQ ID NO: 2, 4, 14, or 16. The polypeptide may comprise, e.g., at least about 10, 12, or 14 contiguous amino acids of SEQ ID NO: 2, 4, 14, or 16; and/or may have a sequence identity of, e.g., at least about 65%, preferably 70-75%, more preferably 80-85%, even more preferably 90-95% or most preferably 97-99% to SEQ ID NO: 2, 4, 14, 16 or a fragment thereof; and/or may comprise a sequence that is substantially homologous to SEQ ID NO: 2, 4, 14, 16 or a fragment thereof. The polypeptide may further comprise a heterologous sequence; may exhibit a PDE2 activity; may be from a mammal, e.g., a human, mouse or rat; and/or may be substantially purified.

In another aspect, the invention relates to an isolated polynucleotide which comprises the nucleotide sequence of SEQ ID NO: 1, 3, 13, 15 or a fragment or variant of SEQ ID NO: 1, 3, 13, 15 or a complement thereof. The polynucleotide many comprise; e.g., at least about 8, 10, 12, 14, or 15 contiguous nucleotides of SEQ ID NO: 1, e.g., about 15 continuous nucleotides. The polynucleotide may further comprise a heterologous sequence; and/or may be from a mammal, e.g., a human, mouse or rat; and/or may be DNA, cDNA, RNA, PNA or combinations thereof. The polynucleotide may comprise a sequence that hybridizes to SEQ ID NO: 1, 3, 13, 15 or a fragment thereof under conditions of high stringency; and/or may comprise a sequence that is substantially homologous to SEQ ID NO: 1, 3, 13, 15 or a fragment thereof; and/or may have a sequence identity of, e.g., at least about 65%, preferably 70-75%, more preferably 80-85%, even more preferably 90-95% or most preferably 97-99% to SEQ ID NO: 1, 3, 13, 15 or a fragment thereof. In another aspect, the invention relates to an isolated polynucleotide which comprises a nucleotide sequence that codes without interruption for the polypeptide of SEQ ID NO: 2, 4, 14, 16, or which comprises a nucleotide sequence that codes without interruption for a fragment or variant of the polypeptide of SEQ ID NO: 2, 4, 14, or 16; or a complement thereof.

In another aspect, the invention relates to a recombinant construct comprising a polynucleotide as above, which may be operatively linked to a regulatory sequence, e.g., wherein said construct comprises a baculovirus expression vector. The invention also relates to a cell comprising such a construct, e.g., a mammalian, human, yeast or insect cell, preferably an SF9 cell.

The invention also relates to a method of making such a cell, comprising introducing a construct or polynucleotide as above into a cell. The invention also relates to a method to make a polypeptide of the invention, comprising incubating a cell as above under conditions in which the polypeptide is expressed, and harvesting the polypeptide.

In another aspect, the invention relates to an antibody, antigen-specific antibody fragment, or other specific binding partner, which is specific for a polypeptide of the invention, e.g., wherein said antibody, antigen-specific antibody fragment, or specific binding partner is specific for the polypeptide of SEQ ID NO: 2, 4, 14, or 16.

In another aspect, the invention relates to methods of diagnosis, e.g., a method to determine the presence of a disease or condition, or a susceptibility to a disease or condition in a patient in need thereof, where said disease or condition is associated with an over- or underexpression of a polynucleotide (e.g., mRNA) of the invention, comprising contacting a cell, tissue, cell extract, or nucleic acid of said patient with a polynucleotide as above, and/or determining the amount or level of said nucleic acid. The cell or nucleic acid may be from the brain of said patient, e.g., from the hippocampus, and may be from a neuron.

The invention also relates to a method of diagnosis, comprising determining a mutation or polymorphism or SNP in the genome of a cell, wherein said mutation occurs in the nucleotide sequence of SEQ ID NO: 1, 3, 13, or 15, or in the sequence of a polynucleotide which encodes a polypeptide of SEQ ID NO: 2, 4, 14, or 16, or in regulatory or untranslated regions thereof (e.g., in a promoter region).

The invention also relates to a method to determine the presence of a disease or condition or a susceptibility to a disease or condition, wherein said condition is associated or mediated by PDE, e.g., such as an over- or under-expression of, or activity of, a polypeptide of the invention, comprising contacting a cell, tissue or cell extract of said patient with an antibody which is specific for a polypeptide of the invention, and detecting the amount or activity of said polypeptide. An example of a disease associated with over-expression of PDE2 is a cancer, such as gastrointestinal stromal tumor (Frolov et al., Mol. Cancer Ther., 2:699-709, 2003).

The invention also relates to a method to determine the presence of a disease or condition or susceptibility to a disease or condition, wherein said disease or condition is associated with a mutated PDE2A2 or PDE2A4, comprising identifying such a mutation in a PDE2A2 or PDE2A4 isolated from a patient

In another aspect, the invention relates to methods to screen for agents that modulate (e.g., stimulate or inhibit) expression or activity of a polypeptide of the invention, or of a polynucleotide which encodes it, comprising contacting a cell, preferably from neuronal tissue, or a tissue cell extract with a putative modulatory agent, and measuring the amount or activity of said polypeptide or polynucleotide, or monitoring cAMP or cGMP levels. Preferably, for high-throughput screening, the activity of the polypeptide or polynucleotide may be measured with a fluorescent compound-labelled cGMP or cAMP substrate, wherein the hydrolysis of the substrate may be measured by monitoring the increase in fluorescence polarization. Alternatively, the invention relates to methods to screen for agents which bind to a polypeptide or polynucleotide of the invention, comprising contacting a polypeptide or polynucleotide with a putative binding agent and determining the presence of a bound complex (e.g., a nucleic acid hybrid, antigen-antibody complex, protein-protein interaction, ligand-target complex, or the like). Methods of the invention can be performed in vitro, ex vivo, or in vivo.

In another aspect, the invention relates to a transgenic animal (e.g., a mouse) comprising at least one copy of a PDE2A4 or PDE2A2 polynucleotide of the invention, wherein the animal over expresses functional PDE2A4 or PDE2A2, or a functional fragment or analog thereof, compared to a non-transgenic animal. In another aspect, the invention relates to a knockout animal, e.g., a mouse, whose genome lacks a gene expressing a functional PDE2A4 or PDE2A2 or PDE2A2 or functional fragment or variant thereof; or to a transgenic animal in which the natural PDE2A4 or PDE2A2 is replaced by a heterologous transgenic (e.g., human) PDE2A4 or PDE2A2.

In another aspect, the invention relates to a pharmaceutical composition comprising a polypeptide or polynucleotide of the invention and a pharmaceutically acceptable carrier. In another aspect, the invention relates to a prophylactic or therapeutic method of treating a disease condition mediated by, or associated with, aberrant expression and/or activity of PDE2A4 or PDE2A2, comprising administering to a patient in need thereof an agent which modulates the expression and/or activity of said PDE2A4 or PDE2A2.

Polypeptides

PDE2As of the invention belong to a family of phosphodiesterases (PDEs) that catalyze the hydrolysis of nucleoside monophosphates (including cAMP and cGMP). These cyclic nucleotides act as second messengers within cells, and carry impulses from cell surface receptors to which are bound, e.g., various hormones and neurotransmitters. Phosphodiesterases degrade these cyclic mononucleotides once their messenger role is completed, and thereby regulate the level of cyclic nucleotides within cells and maintain cyclic nucleotide homeostasis. A subclass of PDEs, designated PDE2s, are characterized by, e.g., ability to hydrolyze both cAMP and cGMP, a lower Michaelis constant for cGMP than for cAMP, and sensitivity to certain drugs, such as EHNA.

A BLAST search of the human genomic database reveals that human PDE2A4 cDNA sequences are localized on two genomic clones, clone RP11-796A3 (AC055829) and clone RP11-169D4 (AP005019). Human PDE2A4 protein is coded by 31 exons with the first two exons on clone RP11-796A3 and the rest of the exons on clone RP11-169D4.

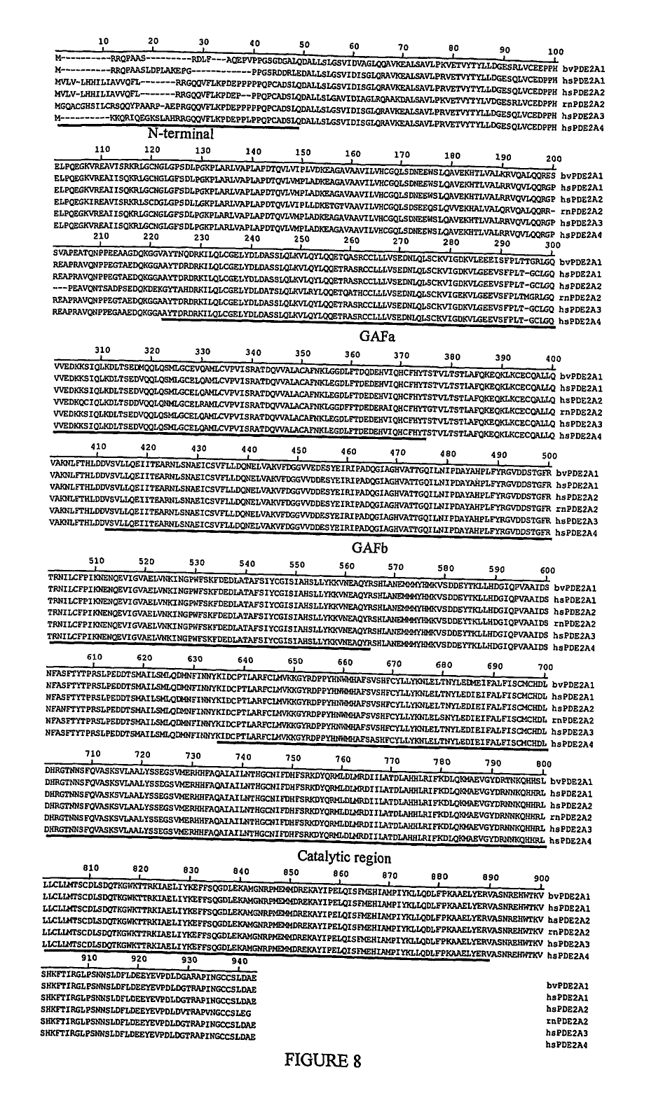

FIG. 8 illustrates functional domains of the PDE2As of the present invention. These include, but are not limited to, e.g., GAFa (involved in dimerization), GAFb (a cGMP binding domain), and a catalytic region (in the C-terminal half of the molecule) which is conserved in all known PDEs. The present invention includes variants of the disclosed polypeptide sequences which comprise mutations in one or more functional domains identified in FIG. 8, and regions between these domains. Amino acid substitutions can be conservative or non-conservative. Comparing between the various isoforms provides guidance on what regions can be mutated without eliminating the activity of the polypeptide (e.g., amino acid positions that are not conserved between two or more isoforms, such as positions 61, 62, 65, 92, 115, 259, 295, 363, 367, etc.).

A polypeptide of the present invention may be a recombinant polypeptide, a natural polypeptide or a synthetic or semi-synthetic polypeptide, or combinations thereof, preferably a recombinant polypeptide. As used herein, the terms polypeptide, oligopeptide and protein are interchangeable.

The polypeptides of the present invention are preferably provided in an isolated form, and may be purified, e.g. to homogeneity. The term “isolated,” when referring, e.g., to a polypeptide or polynucleotide, means that the material is removed from its original environment (e.g., the natural environment if it is naturally occurring), and isolated or separated from at least one other component with which it is naturally associated. For example, a naturally-occurring polypeptide present in its natural living host is not isolated, but the same polypeptide, separated from some or all of the coexisting materials in the natural system, is isolated. Such polypeptides could be part of a composition, and still be isolated in that such composition is not part of its natural environment A polypeptide can also be free, or substantially free of, any proteins or other contaminants derived from the source it is originally isolated. Such polypeptides are generally recombinantly made, and therefore can be distinguished from a polypeptide isolated from its natural source.

The term “fragment” or “variant,” when referring to a polypeptide of the invention means a polypeptide which retains substantially at least one of the biological functions or activities of the polypeptide. Such a biological function or activity can be, e.g., any of those described above, and includes having the ability to react with an antibody, i.e., having an epitope-bearing peptide. Fragments or variants of the polypeptides, e.g. of SEQ ID NO: 2 or 14, have sufficient similarity to those polypeptides so that at least one activity of the native polypeptides is retained. Fragments or variants of smaller polypeptides, e.g., of the polypeptide of SEQ ID NO: 4 or 16, retain at least one activity (e.g., an activity expressed by a functional domain thereof, or the ability to react with an antibody or antigen-binding fragment of the invention) of a comparable sequence found in the native polypeptide.

Polypeptide fragments of the invention may be of any size that is compatible with the aspects of the invention. They may range in size from the smallest specific epitope (e.g., about 6 amino acids) to a nearly full-length gene product (e.g., a single amino acid shorter than SEQ ID NO: 2).

Fragments of the polypeptides of the present invention may be employed, e.g., for producing the corresponding full-length polypeptide by peptide synthesis, e.g., as intermediates for producing the full-length polypeptides; for inducing the production of antibodies or antigen-binding fragments; as “query sequences” for the probing of public databases, or the like.

A variant of a polypeptide of the invention may be, e.g., (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (ii) one in which the polypeptide is fused with another compound, such as a compound to increase the half-life of the polypeptide (for example, polyethylene glycol), or (iv) one in which additional amino acids are fused to the polypeptide, such as a leader or secretory sequence or a sequence which is employed for purification of the polypeptide, commonly for the purpose of creating a genetically engineered form of the protein that is susceptible to, secretion from a cell, such as a transformed cell. The additional amino acids may be from a heterologous source, or may be endogenous to the natural gene.

Variant polypeptides belonging to type (i) above include, e.g., muteins, analogs and derivatives. A variant polypeptide can differ in amino acid sequence by, e.g., one or more additions, substitutions, deletions, insertions, inversions, fusions, and truncations or a combination of any of these.

Variant polypeptides belonging to type (ii) above include, e.g., modified polypeptides. Known polypeptide modifications include, but are not limited to, glycosylation, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formatin, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in many basic texts, such as Proteins—Structure and Molecular Properties, 2nd ed., T. E. Creighton, W.H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al., Meth. Enzynol., 182:626-46 (1990) and Rattan et al., Ann. N.Y. Acad. Sci., 663:48-62 (1992).

Variant polypeptides belonging to type (iii) are well known in the art and include, e.g., PEGulation or other chemical modifications.

Variants polypeptides belonging to type (iv) above include, e.g., preproteins or proproteins which can be activated by cleavage of the proprotein portion to produce an active mature polypeptide. Variants include a variety of hybrid, chimeric or fusion polypeptides. Typical examples of such variants are discussed elsewhere herein.

Many other types of variants are known to those of skill in the art. For example, as is well known, polypeptides are not always entirely linear. For instance, polypeptides may be branched as a result of ubiquitination, and they may be circular, with or without branching, generally as a result of post-translation events, including natural processing events and events brought about by human manipulation which do not occur naturally. Circular, branched and branched circular polypeptides may be synthesized by non-translational natural processes and by synthetic methods.

Modifications or variations can occur anywhere in a polypeptide, including the peptide backbone, the amino acid side-chains and the amino or carboxyl termini. The same type of modification may be present in the same or varying degree at several sites in a given polypeptide. Also, a given polypeptide may contain more than one type of modification. Blockage of the amino or carboxyl group in a polypeptide, or both, by a covalent modification, is common in naturally occurring and synthetic polypeptides. For instance, the amino terminal residue of polypeptides made in E. coli, prior to proteolytic processing, is often N-formylmethionine. The modifications can be a function of how the protein is made. For recombinant polypeptides, for example, the modifications are determined by the host cell posttranslational modification capacity and the modification signals in the polypeptide amino acid sequence. Accordingly, when glycosylation is desired, a polypeptide can be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

Variant polypeptides can be fully functional or can lack function in one or more activities, e.g., in any of the functions or activities described above. Among the many types of useful variations are, e.g., those which exhibit alteration of catalytic activity. For example, one embodiment involves a variation at the binding site that results in binding but not hydrolysis, or slower hydrolysis, of cAMP or cGMP. A further useful variation at the same site can result in altered affinity for cAMP or cGMP. Useful variations also include changes that provide for affinity for another cyclic nucleotide. Another useful variation includes one that prevents activation by protein kinase A. Another useful variation provides a fusion protein in which one or more domains or subregions are operationally fused to one or more domains or subregions from another phosphodiesterase isoform or family.

As noted above, the polypeptides of the present invention include, e.g., isolated polypeptides comprising the sequences of SEQ ID NO: 2 or 14 (in particular the mature polypeptide) and fragments thereof. The polypeptides of the invention also include polypeptides which have varying degrees of sequence identity thereto, so long as such polypeptides contain a sequence (e.g., at their N-terminal ends) that is substantially homologous to the amino acid sequence of SEQ ID NO: 4 or 16, or that shows substantial sequence homology (sequence identity) to it. Sequence identity can be measured along the entire length of the polypeptide (or nucleic acid), and comprise at last about 65%, 70-75% or 80-85% sequence identity thereto, and most preferably about 90-95% or about 97-99% sequence identity thereto. The invention also encompasses polypeptides having a lower degree of sequence identity, but having sufficient similarity so as to perform one or more of the functions or activities exhibited by the phosphodiesterase.

In accordance with the present invention, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:
Percent Identity=100 [1−(C/R)]
wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence.

The description herein for percent identity or percent homology is intended to apply equally to nucleotide or amino acid sequences

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

A preferred, non-limiting example of such a mathematical algorithm is described in Karlin et al., Proc. Natl. Acad. Sci. USA, 90:5873-5877 (1993). Such an algorithm is incorporated into the NBLAST and XBLAST programs (version 2.0) as described in Altschul et al., Nucleic Acids Res., 25:3389-3402 (1997). When utilizing BLAST and Gapped BLAST programs, the default parameters of the respective programs (e.g., NBLASST) can be used. In one embodiment, parameters for sequence comparison can be set at score=100, wordlength-12, or can be varied (e.g., W=5 or W=20).

In a preferred embodiment, the percent identity between two amino acid sequences is determined using the Needleman et al. (J. Mol. Biol., 48:444-453 (1970)) algorithm which has been incorporated into the GAP program in the GCG software package using either a BLOSUM 62 matrix or a PAM250 matrix, and a gap weight of 16, 14, 12, 10, 8, 6, or 4 and a length weight of 1, 2, 3, 4, 5 or 6. In yet another preferred embodiment, the percent identity between two nucleotide sequences is determined using the GAP program I the GCG software package (Devereux et al., Nucleic Acids Res., 12 (1):387 (1984)) using a NWSgapdna. CMP matrix and a gap weight of 40, 50, 60, 70, or 80 and a length weight of 1, 2, 3, 4, 5 or 6.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis et al., Comput. Appl. Biosci., 10:3-5 (1994); and FASTA described in Pearson et al., Proc. Natl. Acad. Sci. USA, 85:2444-8 (1988).

Polypeptides, and fragments or variants thereof, within the present invention may also contain unbroken stretches of amino acids containing fewer than the full-length amino acids of SEQ ID NO: 2, 4, 14, or 16 disclosed herein, e.g., about 6, 8, 10, 12, 14, 15, 20, 25, 30, 35, 40, 50, 60, 70, 80 or 84 amino acids, preferably at least about 60 amino acids.

As used with respect to the polypeptides (and polynucleotides) of the present invention, the term fragment refers to a sequence that is a subset of a larger sequence (i.e., a continuous or unbroken sequence of residues within a larger sequence). Thus, for example, 15 residues of a novel 15-mer disclosed herein can contain a total of 6 fragments of 10 residues each (e.g. 1-10, 2-11, 3-12, 4-13, 5-14, and 6-15). 10-mers or larger peptides already present in the art are, of course, excluded.

The polypeptides, and fragments thereof, of the present invention may be found in the cells and tissues of any species of animal, but are preferably found in cells from mammals, e.g., mouse, rat, rabbit, farm animals, pets, primates, etc., especially the cells of humans. In any given animal, the polypeptides and fragments thereof within the present invention may be found in a variety of tissues. Methods of determining the tissue or cellular location of such polypeptides are conventional and include, e.g., conventional methods of immunohistochemistry. Various PDEs are found in, e.g., heart, ovary, pancreas, kidney, breast, liver, testis, prostate, skeletal muscle, and osteoblasts. See, e.g., Beavo, Physiological Reviews 75:725-748 (1995) and U.S. Pat. No. 5,798,246. Specific isoforms often exhibit tissue specificity. For example, the PDE2A4 protein is highly expressed in placenta. The PDE2A4 protein is also localized in heart, brain, lung, liver, skeletal muscle, kidney, and pancreas.

Nucleic Acids

As discussed above, the invention includes, e.g., cDNAs encoding full length polypeptides of the invention, and fragments from the 5′-terminal regions thereof, represented by SEQ ID NO: 1 and 13.

The polynucleotide sequence of SEQ ID NO: 1 contains an open reading frame (or ORF) coding for the polypeptide of SEQ ID NO: 2 at nucleotides 141-2939 (with nucleotides 2937-2939 representing the “TGA” termination codon).

The polynucleotide sequence of SEQ ID NO: 13 contains an open reading frame (or ORF) coding for the polypeptide of SEQ ID NO: 14 at nucleotides 48-2852 (with nucleotides 2850-2852 representing the “TGA” termination codon).

As used herein, the phrase “an isolated polynucleotide which is SEQ ID NO,” or “an isolated polynucleotide which is selected from SEQ ID NO,” refers to an isolated nucleic acid molecule from which the recited sequence was obtained (i.e., the mRNA). Because of sequencing errors, typographical errors, etc., the actual naturally occurring sequence may differ from a SEQ ID listed herein. Thus, the phrase indicates the specific molecule from which the sequence was derived, rather than a molecule having that exact recited nucleotide sequence, analogously to how a culture depository number refers to a specific cloned fragment in a cryotube.

A polynucleotide of the present invention may be a recombinant polynucleotide, a natural polynucleotide, or a synthetic or semi-synthetic polynucleotide, or combinations thereof. As used herein, the terms polynucleotide, oligonucleotide, oligomer and nucleic acid are interchangeable.

As used herein, the term “gene” means a segment of DNA involved in producing a polypeptide chain; it may include regions preceding and following the coding region (leader and trailer) as well as intervening sequences (introns) between individual coding segments (exons). Of course, cDNAs lack the corresponding introns. The invention includes isolated genes (e.g., genomic clones) which encode polypeptides of the invention.

Polynucleotides of the invention may be RNA, PNA, or DNA, e.g., cDNA, genomic DNA, and synthetic or semi-synthetic DNA, or combinations thereof. The DNA may be triplex, double-stranded or single-stranded, and if single stranded, may be the coding strand or non-coding (anti-sense) strand. It can comprise hairpins or other secondary structures. The RNA includes oligomers (including those having sense or antisense strands), mRNAs (e.g., having the alternative splices of PDE2A4), polyadenylated RNA, total RNA, single strand or double strand RNA, or the like. DNA/RNA duplexes are also encompassed by the invention.

The polynucleotides and fragments thereof of the present invention may be of any size that is compatible with the invention, e.g., of any desired size that is effective to achieve a desired specificity when used as a probe. Polynucleotides may range in size, e.g., from the smallest specific probe (e.g., about 10-12 nucleotides) to greater than a full-length cDNA, e.g., in the case of a fusion polynucleotide or a polynucleotide that is part of a genomic sequence; fragments may be as large as, e.g., one nucleotide shorter than a full-length cDNA.

A fragment of a polynucleotide according to the invention may be used, e.g., as a hybridization probe, as discussed elsewhere herein.

Many types of variants of polynucleotides are encompassed by the invention including, e.g., (i) one in which one or more of the nucleotides is substituted with another nucleotide, or which is otherwise mutated; or (ii) one in which one or more of the nucleotides is modified, e.g., includes a subtituent group; or (iii) one in which the polynucleotide is fused with another compound, such as a compound to increase the half-life of the polynucleotide; or (iv) one in which additional nucleotides are covalently bound to the polynucleotide, such a sequences encoding a leader or secretory sequence or a sequence which is employed for purification of the polypeptide. The additional nucleotides may be from a heterologous source, or may be endogenous to the natural gene.

Polynucleotide variants belonging to type (i) above include, e.g., polymorphisms, including single nucleotide polymorphisms (SNPs), and mutants. Variant polynucleotides can comprise, e.g., one or more additions, insertions, deletions, substitutions, transitions, transversions, inversions, chromosomal translocations, variants resulting from alternative splicing events, or the like, or any combinations thereof.

A coding sequence which encodes a polypeptide (e.g., a mature polypeptide) of the invention may be identical to the coding sequence shown in SEQ ID NO: 1 or 13 or a fragment thereof, or may be a different coding sequence, which coding sequence, as a result of the redundancy or degeneracy of the genetic code, encodes the same polypeptide as the DNA of SEQ ID NO: 1, 13, or a fragment thereof. Such a peptide is sometimes referred to herein as a “degenerate variant.” Alternatively, the coding sequence may encode a polypeptide that is substantially homologous to the polypeptide of SEQ ID NO: 2 or 14, or a fragment thereof.

A polynucleotide of the invention may have a coding sequence which is a naturally or non-naturally occurring allelic variant of a coding sequence encompassed by the sequence in SEQ ID NO: 1 or 13. As known in the art, an allelic variant is an alternate form of a polynucleotide sequence, which may have a substitution, deletion or addition of one or more nucleotides, which in general does not substantially alter the function of the encoded polypeptide.

Other variant sequences, located in a coding sequence or in a regulatory sequence, may affect (enhance or decrease) the production of, or the function or activity of, a polypeptide of the invention.

Polynucleotide variants belonging to type (ii) above include, e.g., modifications such as the attachment of detectable markers (avidin, biotin, radioactive elements, fluorescent tags and dyes, energy transfer labels, energy-emitting labels, binding partners, etc.) or moieties which improve expression, uptake, cataloging, tagging, hybridization, detection, and/or stability. The polynucleotides can also be attached to solid supports, e.g., nitrocellulose, magnetic or paramagnetic microspheres (e.g., as described in U.S. Pat. No. 5,411,863; U.S. Pat. No. 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967; 5,476,925; and 5,478,893.

Polynucleotide variants belonging to type (iii) above are well known in the art and include, e.g., various lengths of polyA⁺ tail, 5′cap structures, and nucleotide analogs, e.g., inosine, thionucleotides, or the like.

Polynucleotide variants belonging to type (iv) above include, e.g., a variety of chimeric, hybrid or fusion polynucleotides. For example, a polynucleotide of the invention can comprise a coding sequence and additional non-naturally occurring or heterologous coding sequence (e.g., sequences coding for leader, signal, secretory, targeting, enzymatic, fluorescent, antibiotic resistance, and other functional or diagnostic peptides); or a coding sequence and non-coding sequences, e.g., untranslated sequences at either a 5′ or 3′ end, or dispersed in the coding sequence, e.g., introns.

More specifically, the present invention includes polynucleotides wherein the coding sequence for the polypeptide (e.g., a mature polypeptide) is fused in the same reading frame to a polynucleotide sequence (e.g., a heterologous sequence), e.g. one which aids in expression and secretion of a polypeptide from a host cell, for example, a leader sequence which functions as a secretory sequence for controlling transport of a polypeptide from the cell and/or a transmembrane anchor which facilitates attachment of the polypeptide to a cellular membrane. A polypeptide having a leader sequence is a preprotein and may have the leader sequence cleaved by the host cell to form a mature form of the polypeptide. The polynucleotides may also encode for a proprotein which is the mature protein plus additional N-terminal amino acid residues. A mature protein having a prosequence is a proprotein and is generally an inactive form of the protein. Once the prosequence is cleaved an active protein remains.

Polynucleotides of the present invention may also have a coding sequence fused in frame to a marker sequence that allows for identification and/or purification of the polypeptide of the present invention. The marker sequence may be, e.g., a hexa-histidine tag (e.g., as supplied by a pQE-9 vector) to provide for purification of the mature polypeptide fused to the marker in the case of a bacterial host, or, for example, the marker sequence may be a hemagglutinin (HA) tag when a mammalian host, e.g. COS-7 or QM7 cells, is used. The HA tag corresponds to an epitope derived from the influenza hemagglutinin protein (Wilson et al., Cell, 37:767 (1984)).

Other types of polynucleotide variants will be evident to one of skill in the art. For example, the nucleotides of a polynucleotide can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, etc.

Also, polynucleotides of the invention may have a coding sequence derived from another genetic locus of an organism, providing it has a substantial homology to, e.g., part or all of the sequence of SEQ ID NO: 1 or 13 or from another organism (e.g., an ortholog).

Of course, it is understood that variants exclude any sequences disclosed prior to the invention.

Polynucleotides according to the present invention can be labeled according to any desired method. The polynucleotide can be labeled using radioactive tracers such as, e.g., ³²P, ³⁵S, ³H, or ¹⁴C. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a polynucleotide of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

The present invention includes polynucleotides encoding all of the polypeptides and fragments or variants thereof, as disclosed hereinabove, provided that they incorporate therein a close homolog, or a fragment thereof, of a polynucleotide encoding a 14-mer or 17-mer of the present invention, or a fragment or variant thereof. For example, a polynucleotide of the invention may comprise a sequence which has a sequence identity of at least about 65-100%, (e.g., at least about 70-75%, 80-85%, 90-95% or 97-99%) to, or which is substantially homologous to, or which hybridizes under conditions of high stringency to, the nucleotide sequence of SEQ ID NO: 3 or 15, or to a fragment thereof; or which is complementary to one of those sequences.

The term “substantially homologous,” when referring to polynucleotide sequences, means that the nucleotide sequences are at least about 90-95%, preferably 97-99%, or more identical. In accordance with the present invention, the term “substantially homologous,” when referring to a protein sequence, means that the amino acid sequences are at least about 90-95% or 97-99% or more identical. A substantially homologous amino acid sequence of the invention can be encoded by a nucleic acid sequence hybridizing to the nucleic acid sequence, or portion thereof, of the sequence shown in SEQ ID NO: 1, 3, 13, or 15 under conditions of high stringency.

Conditions of “high stringency,” as used herein, means, for example, incubating a blot overnight (e.g., at least 12 hours) with a long polynucleotide probe in a hybridization solution containing, e.g., about 5×SSC, 0.5% SDS, 100 μg/ml denatured salmon sperm DNA and 50% formamide, at 42° C. Blots can be washed at high stringency conditions that allow, e.g., for less than 5% bp mismatch (e.g., wash twice in 0.1×SSC and 0.1% SDS for 30 min at 65° C.), thereby selecting sequences having, e.g., 95% or greater sequence identity.

Other non-limiting examples of high stringency conditions include a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a 1% SDS solution at 42° C. Whereas high stringency washes can allow for less than 5% mismatch, reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

Constructs

The present invention also relates to recombinant constructs that contain vectors plus polynucleotides of the present invention. Such constructs comprise a vector, such as a plasmid or viral vector, into which a polynucleotide sequence of the invention has been inserted, in a forward or reverse orientation.

Large numbers of suitable vectors are known to those of skill in the art, and many are commercially available. The following vectors are provided by way of example; Bacterial: pQE70, pQE60, pQE-9 (Qiagen), pBS, pD10, phagescript, psiX174, pBluescript SK, pBSKS, pNH8A, pNH16a, pNH18A, pNH46A (Stratagene); pTRC99a, pKK223-3, pKK233-3, pDR540, pRIT5 (Pharmacia); Eukaryotic: pWLNEO, pSV2CAT, pOG44, pXT1, pSG (Stratagene) pSVK3, pBPV, pMSG, pSVL (Pharmacia). However, any other plasmid or vector may be used as long as it is replicable and viable in the host.

In a preferred embodiment, the vector is an expression vector, into which a polynucleotide sequence of the invention is inserted so as to be operatively linked to an appropriate expression control (regulatory) sequence(s) (e.g., promoters and/or enhancers) which directs mRNA synthesis. Appropriate expression control sequences, e.g., regulatable promoter or regulatory sequences known to control expression of genes in prokaryotic or eukaryotic cells or their viruses, can be selected for expression in prokaryotes (e.g., bacteria), yeast, plants, mammalian cells or other cells. Preferred expression control sequences are derived from highly-expressed genes, e.g., from operons encoding glycolytic enzymes such as 3-phosphoglycerate kinase (PGK), α-factor, acid phosphatase, or heat shock proteins, among others. Such expression control sequences can be selected from any desired gene, e.g using CAT (chloramphenicol transferase) vectors or other vectors with selectable markers. Two appropriate vectors for such selection are pKK232-8 and pCM7.

Particular named bacterial promoters which can be used include lacI, lacZ, T3, T7, gpt, lambda P_{R}, P_{L} and trp. Eukaryotic promoters include CMV immediate early, HSV thymidine kinase, early and late SV40, adenovirus promoters, LTRs from retrovirus, and mouse metallothionein-I. Selection of the appropriate vector and promoter is well within the level of ordinary skill in the art.

Transcription of the DNA encoding the polypeptides of the present invention by higher eukaryotes can be increased by inserting an enhancer sequence into the expression vector. Enhancers are cis-acting elements of DNA, usually about from 10 to 300 bp that act on a promoter to increase its transcription. Representative examples include the SV40 enhancer on the late side of the replication origin bp 100 to 270, a cytomegalovirus early promoter enhancer, the polyoma enhancer on the late side of the replication origin, and adenovirus enhancers.

Generally, recombinant expression vectors also include origins of replication. An expression vector may contain a ribosome binding site for translation initiation, a transcription termination sequence, a polyadenylation site, splice donor and acceptor sites, and/or 5′ flanking or non-transcribed sequences. DNA sequences derived from the SV40 splice and polyadenylation sites may be used to provide required nontranscribed genetic elements. The vector may also include appropriate sequences for amplifying expression. In addition, expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

Large numbers of suitable expression vectors are known to those of skill in the art, and many are commercially available. Suitable vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, adeno-associated virus, TMV, fowl pox virus, and pseudorabies. However, any other vector may be used as long as it is replicable and viable in a host. Appropriate cloning and expression vectors for use with prokaryotic and eukaryotic hosts are described, e.g., by Sambrook, et al., Molecular Cloning: A Laboratory Manual, Second Edition, Cold Spring Harbor, N.Y., (1989), Wu et al., Methods in Gene Biotechnology (CRC Press, New York, N.Y., 1997), Recombinant Gene Expression Protocols, in Methods in Molecular Biology, Vol. 62, (Tuan, ed., Humana Press, Totowa, N.J., 1997), and Current Protocols in Molecular Biology, (Ausabel et al., Eds.), John Wiley & Sons, NY (1994-1999).

In an embodiment, a Baculovirus-based expression system is used. Baculoviruses represent a large family of DNA viruses that infect mostly insects. The prototype is the nuclear polyhedrosis virus (AcMNPV) from Autographa californica, which infects a number of lepidopteran species. One advantage of the baculovirus system is that recombinant baculoviruses can be produced in vivo. Following co-transfection with transfer plasmid, most progeny tend to be wild type and a good deal of the subsequent processing involves screening. To help identify plaques, special systems are available that utilize deletion mutants. By way of non-limiting example, a recombinant AcMNPV derivative (called BacPAK6) has been reported in the literature that includes target sites for the restriction nuclease Bsu36I upstream of the polyhedrin gene (and within ORF 1629) that encodes a capsid gene (essential for virus viability). Bsf361 does not cut elsewhere in the genome and digestion of the BacPAK6 deletes a portion of the ORF 1629, thereby rendering the virus non-viable. Thus, with a protocol involving a system like Bsu361-cut BacPAK6 DNA most of the progeny are non-viable so that the only progeny obtained after co-transfection of transfer plasmid and digested BacPAK6 is the recombinant because the transfer plasmid, containing the exogenous DNA, is inserted at the Bsu361 site thereby rendering the recombinants resistant to the enzyme. See Kitts and Possee, A method for producing baculovirus expression vectors at high frequency, BioTechniques, 14, 810-817 (1993). For general procedures, see King and Possee, The Baculovirus Expression System: A Laboratory Guide, Chapman and Hall, New York (1992) and Recombinant Gene Expression Protocols, in Methods in Molecular Biology, Vol. 62, (Tuan, ed., Humana Press, Totowa, N.J., 1997), at Chapter 19, pp. 235-246.

Appropriate DNA sequences may be inserted into a vector by any of a variety of procedures. In general, the DNA sequence is inserted into an appropriate restriction endonuclease site(s) by procedures known in the art. Such procedures and others are deemed to be within the scope of those skilled in the art. Conventional procedures for this and other molecular biology techniques discussed herein are found in many readily available sources, e.g., Sambrook, et al., Molecular Cloning: A Laboratory Manual, Second Edition, Cold Spring Harbor, N.Y., (1989). If desired, a heterologous structural sequence is assembled in an expression vector in appropriate phase with translation initiation and termination sequences, and preferably, a leader sequence capable of directing secretion of translated protein into the periplasmic space or extracellular medium.

Transformed Cells and Methods of Producing Polypeptides of the Invention

The present invention also relates to host cells which are transformed/transfected/transduced with constructs such as those described above, and to progeny of said cells, especially where such cells result in a stable cell line that can be used for assays of PDE2A activity, e.g., in order to identify agents which modulate PDE2A activity, and/or for production (e.g., preparative production) of the polypeptides of the invention.

As representative examples of appropriate hosts, there may be mentioned: bacterial cells, such as E. coli, Streptomyces, Salmonella typhimurium; fungal cells, such as yeast; insect cells such as Drosophila 52 and Spodoptera Sf9 (and other insect expression systems); animal cells, including mammalian cells such as CHO, QT6 (ATCC CRL-1708), QM7 (ATCC CRL-1962), COS (e.g., the COS-7 lines of monkey kidney fibroblasts described by Gluzman, Cell, 23:175 (1981)), C127, 3T3, CHO, HeLa, BHK or Bowes melanoma cell lines; plant cells, etc. The selection of an appropriate host is deemed to be within the knowledge of those skilled in the art based on the teachings herein. Cell lines used for testing putative modulatory agents are commonly mammalian cells whose cAMP or cGMP levels are monitored for indications of varying phosphodiesterase (PDE2A) activity.

In a most preferred embodiment, the host cells are insect cells of Spodoptera species, most especially SF9 cells, from Spodoptera frugiperda. Polypeptides (e.g., full length polypeptides) of the present invention are readily obtainable from insect cells using a baculovirus expression vector. Such expression is readily characterized using methods well known in the art. See, e.g., Wang et al, Expression, Purification, and Characterization of Human cAMP-Specific Phosphodiesterase (PDE4) Subtypes A, B, C, and D, Biochem. Biophys. Res. Comm., 234:320-4 (1997).

Introduction of a construct into a host cell can be effected by, e.g., calcium phosphate transfection, DEAE-Dextran mediated transfection, lipofection a gene gun, or electroporation (Davis, L., Dibner, M., Battey, I., Basic Methods in Molecular Biology, (1986)).

Following transformation of a suitable host strain and growth of the host strain to an appropriate cell density, the selected promoter can be induced by appropriate means (e.g., temperature shift or chemical induction) if desired, and cells cultured for an additional period. The engineered host cells are cultured in conventional nutrient media modified as appropriate for activating promoters (if desired), selecting transformants or amplifying the genes of the present invention. The culture conditions, such as temperature, pH and the like, are those previously used with the host cell selected for expression, and will be apparent to the ordinarily skilled artisan.

Cells are typically harvested by centrifugation, disrupted by physical or chemical means, and the resulting crude extract retained for further purification. Alternatively, when a heterologous polypeptide is secreted from the host cell into the culture fluid, supernatants of the culture fluid can be used as a source of the protein. Microbial cells employed in expression of proteins can be disrupted by any convenient method, including freeze-thaw cycling, sonication, mechanical disruption, or use of cell lysing agents, such methods being well known to those skilled in the art.

The polypeptide can be recovered and purified from recombinant cell cultures by conventional methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography, or the like. Protein refolding steps can be used, as necessary, in completing configuration of the mature protein. High performance liquid chromatography (HPLC) can be employed for final purification steps. See, e.g., Salanova et al, Heterologous Expression and Purification of Recombinant Rolipram-Sensitive Cyclic AMP-Specific Phosphodiesterases, in Methods: A Companion to Methods in Enzymology, 14:55-64 (1998).

In addition to the methods described above for producing polypeptides recombinantly from a prokaryotic or eukaryotic host, polypeptides of the invention can be prepared from natural sources, or can be prepared by chemical synthetic procedures (e.g., synthetic or semi-synthetic), e.g., with conventional peptide synthesizers. Cell-free translation systems can also be employed to produce such proteins using RNAs derived from the DNA constructs of the present invention. Proteins of the invention can also be expressed in, and isolated and/or purified from, transgenic animals or plants. Procedures to make and use such transgenic organisms are conventional in the art. Some such procedures are described elsewhere herein.

Antibodies, Antigen-Binding Fragments or Other Specific Binding Partners

The polypeptides, their fragments or variants thereof, or cells expressing them can also be used as immunogens to produce specific antibodies, or antigen-binding fragments, thereto. By a “specific” antibody or antigen-binding fragment is meant one, which binds selectively (preferentially) to a PDE2A of the invention, or to a fragment or variant thereof, in particular to he N-terminal sequences set forth in FIGS. 2 and 7 (SEQ ID NO: 4 and 16), or fragments and variants thereof. An antibody “specific” for a polypeptide means that the antibody recognizes a defined sequence of amino acids within or including the polypeptide.

Antibodies of the invention can be, for example, polyclonal or monoclonal antibodies. The present invention also includes chimeric, recombinant, single chain, and partially or fully humanized antibodies, as well as Fab fragments, or the product of a Fab expression library, and fragments thereof. The antibodies can be IgM, IgG, subtypes, IgG2A, IgG1, etc. Various procedures known in the art may be used for the production of such antibodies and fragments.

Antibodies generated against the polypeptides corresponding to a sequence of the present invention can be obtained, e.g., by direct injection of the polypeptides into an animal or by administering the polypeptides to an animal, e.g. goat, rabbit, mouse, chicken, etc., preferably a non-human. The antibody so obtained will then bind the polypeptide itself. In this manner, even a sequence encoding only a fragment of the polypeptides can be used to generate antibodies binding the whole native polypeptides. Such antibodies can then be used to isolate the polypeptide from tissue expressing that polypeptide. Antibodies can also be generated by administering naked DNA. See, e.g., U.S. Pat. Nos. 5,703,055; 5,589,466; and 5,580,859.

For preparation of monoclonal antibodies, any technique, which provides antibodies produced by continuous cell line cultures can be used. Examples include, e.g., the hybridoma technique (Kohler and Milstein, Nature, 256:495-7 (1975)), the trioma technique, the human B-cell hybridoma technique (Kozbor et al., Immunology Today, 4:72 (1983)), and the EBV-hybridoma technique to produce human monoclonal antibodies (Cole et al., Monoclonal Antibodies and Cancer Therapy, Alan R. Liss, Inc., pp. 77-96 (1985)).

Techniques described for the production of single chain antibodies (e.g., U.S. Pat. No. 4,946,778) can be adapted to produce single chain antibodies to immunogenic polypeptide products of this invention. Also, transgenic animals may be used to express partially or fully humanized antibodies to immunogenic polypeptide products of this invention.

The invention also relates to other specific binding partners which include, e.g., aptamers and PNA.

Transgenic and Knockout Animals

The invention disclosed herein also relates to a non-human transgenic animal comprising within its genome one or more copies of the polynucleotides encoding the novel polypeptides of the invention. The transgenic animals of the invention may contain within their genome multiple copies of the polynucleotides encoding the polypeptides of the invention, or one copy of a gene encoding such polypeptide but wherein said gene is linked to a promoter (e.g., a regulatable promoter) that will direct expression (preferably overexpression) of said polypeptide within some, or all, of the cells of said transgenic animal. A variety of non-human transgenic organisms are encompassed by the invention, including e.g., drosophila, C. elegans, zebrafish and yeast. The transgenic animal of the invention is preferably a mammal, e.g., a cow, goat, sheep, rabbit, non-human primate, or rat, most preferably a mouse. A transgenic animal in accordance with the present invention can have a phenotype associated with any of the diseases and/or conditions mentioned below, including susceptibility to such a disease or condition. These include, e.g., memory or cognitive dysfunction, cancer susceptibility, etc (see below). The phenotypes can be assessed routinely, e.g., using learning and activity assays, cancer models, long-term potentiation assays (LTP), etc. For general reference on assays for assessing learning and memory in mice, see, e.g., G. C. Tombaugh et al., J. of Neuroscience, 22(22): 9932-9940, 2002.

Methods of producing transgenic animals are well within the skill of those in the art, and include, e.g., homologous recombination, mutagenesis (e.g., ENU, Rathkolb et al., Exp. Physiol., 85(6):635-44, (2000)), and the tetracycline-regulated gene expression system (e.g., U.S. Pat. No. 6,242,667), and will not be described in detail herein. See e.g., Wu et al., Methods in Gene Biotechnology, CRC, pp. 339-366 (1997); Jacenko, O., Strategies in Generating Transgenic Animals, Recombinant Gene Expression Protocols, Vol. 62 of Methods in Molecular Biology, Humana Press, pp. 399-424 (1997).

Transgenic organisms are useful, e.g., for providing a source of a polynucleotide or polypeptide of the invention, or for identifying and/or characterizing agents that modulate expression and/or activity of such a polynucleotide or polypeptide. Transgenic animals are also useful as models for disease conditions related to, e.g., overexpression of a polynucleotide or polypeptide of the invention.

The present invention also relates to a non-human knockout animal whose genome lacks or fails to express a functional PDE2A isoform or functional analog thereof (i.e., the gene is functionally disrupted), such animal commonly being referred to as a “knockout” animal, especially a “knock-out mouse.”

Functional disruption of the gene can be accomplished in any effective way, including, e.g., introduction of a stop codon into any part of the coding sequence such that the resulting polypeptide is biologically inactive (e.g., because it lacks a catalytic domain, a ligand binding domain, etc.), introduction of a mutation into a promoter or other regulatory sequence that is effective to turn it off, or reduce transcription of the gene, insertion of an exogenous sequence into the gene which inactivates it (e.g., which disrupts the production of a biologically-active polypeptide or which disrupts the promoter or other transcriptional machinery), deletion of sequences from the PDE2A gene, etc. Examples of transgenic animals having functionally disrupted genes are well known, e.g., as described in U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824. Knock-outs can be homozygous or heterozygous.

For creating functional disrupted genes, and other gene mutations, homologous recombination technology is of special interest since it allows specific regions of the genome to be targeted. Using homologous recombination methods, genes can be specifically inactivated, specific mutations can be introduced, and exogenous sequences can be introduced at specific sites. These methods are well known in the art, e.g., as described in the patents above. See also, Robertson, Biol. Reproduc., 44(2):238-45, (1991). Generally, the genetic engineering is performed in an embryonic stem (ES) cell, or other pluripotent cell line (e.g., adult stem cells, EG cells), and that genetically-modified cell (or nucleus) is used to create a whole organism. Nuclear transfer can be used in combination with homologous recombination technologies.

For example, a PDE2A locus can be disrupted in mouse ES cells using a positive-negative selection method (e.g., Mansour et al., Nature, 336:348-52 (1988)). In this method, a targeting vector can be constructed which comprises a part of the gene to be targeted. A selectable marker, such as neomycin resistance genes, can be inserted into a PDE2A exon present in the targeting vector, disrupting it. When the vector recombines with the ES cell genome, it disrupts the function of the gene. The presence in the cell of the vector can be determined by expression of neomycin resistance. See, e.g., U.S. Pat. No. 6,239,326. Cells having at least one functionally disrupted gene can be used to make chimeric and germline animals, e.g., animals having somatic and/or germ cells comprising the engineered gene. Homozygous knock-out animals can be obtained from breeding heterozygous knock-out animals. See, e.g., U.S. Pat. No. 6,225,525.

The present invention also relates to a transgenic non-human animal whose, genome comprises one or more genes coding for the human isoform of PDE2A disclosed herein in place of the mammalian gene otherwise coding for said the non-human isoform. A knock-out animal, or animal cell, lacking one or more functional PDE2A genes can be useful in a variety of applications, including as an animal model for a PDE2A-mediated or related condition, for drug screening assays (e.g., for phosphodiesterases other than PDE2A; by making a cell deficient in PDE2A, the contribution of other phosphodiesterases can be specifically examined), as a source of tissues deficient in PDE2A activity, as the starting material for generating an animal in which the endogenous PDE2A is replaced with human PDE2A, and any of the utilities mentioned in any issued U.S. patent on transgenic animals, including, U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824. For instance, PDE2A deficient animal cells can be utilized to study activities related to, e.g., memory formation, inflammation or immuno-modulatory responses. Cells display a variety of enzyme activities which are responsive to extracellular and intracellular signals. By knocking-out phosphodiesterases e.g., one at a time, the physiological pathways using phosphodiesterases can be dissected out and identified.

In addition to the methods mentioned above, transgenic or knock-out animals can be prepared according to known methods, including, e.g., by pronuclear injection of recombinant genes into pronuclei of 1-cell embryos, incorporating an artificial yeast chromosome into embryonic stem cells, gene targeting methods, embryonic stem cell methodology, cloning methods, nuclear transfer methods. See, also, e.g., U.S. Pat. Nos. 4,736,866; 4,873,191; 4,873,316; 5,082,779; 5,304,489; 5,174,986; 5,175,384; 5,175,385; 5,221,778; Gordon et al., Proc. Natl. Acad. Sci., 77:7380-4 (1980); Palmiter et al., Cell, 41:343-5 (1985); Palmiter et al., Ann. Rev. Genet., 20:465-99 (1986); Askew et al., Mol. Cell. Bio., 13:4115-24 (1993); Games et al., Nature, 373:523-7 (1995); Valancius and Smithies, Mol. Cell. Bio., 11:1402-8 (1991); Stacey et al., Mol. Cell. Bio., 14:1009-16 (1994); Hasty et al., Nature, 350:243-6 (1995); Rubinstein et al., Nucl. Acid Res., 21:2613-7 (1993); Cibelli et al., Science, 280:1256-8 (1998). For guidance on recombinase excision systems, see, e.g., U.S. Pat. Nos. 5,626,159, 5,527,695, and 5,434,066. See also Orban, et al., “Tissue-and Site-Specific DNA Recombination in Transgenic Mice,” Proc. Natl. Acad. Sci. USA, 89:6861-5 (1992); O'Gorman, S., et al., “Recombinase-Mediated Gene Activation and Site-Specific Integration in Mammalian Cells,” Science, 251:1351-5 (1991); Sauer et al., “Cre-stimulated recombination at loxP-Containing DNA sequences placed into the mammalian genome,” Polynucleotides Research, 17(1):147-61 (1989); Gagneten et al., Nucl. Acids Res., 25:3326-31 (1997); Xiao and Weaver, Nucl. Acids Res., 25:2985-91 (1997); Agah et al., J. Clin. Invest., 100:169-79 (1997); Barlow et al., Nucl. Acids Res., 25:2543-5 (1997); Araki et al., Nucl. Acids Res., 25:868-72 (1997); Mortensen et al., Mol. Cell. Biol., 12:2391-5 (1992)(G418 escalation method); Lakhlani et al., Proc. Natl. Acad. Sci. USA, 94:9950-5 (1997)(“hit and run”); Westphal and Leder, Curr. Biol., 7:530-3 (1997) (transposon-generated “knock-out” and “knock-in”); Templeton et al., Gene Ther., 4:700-9 (1997) (methods for efficient gene targeting, allowing for a high frequency of homologous recombination events, e.g., without selectable markers); PCT International Publication WO 93/22443 (functionally-disrupted); U.S. Pat. Pub. No. 2003/0121069.

A polynucleotide according to the present invention can be introduced into any non-human animal, including a non-human mammal, mouse (Hogan et al., Manipulating the Mouse Embryo: A Laboratory Manual, Cold Spring Harbor Laboratory, Cold Spring Harbor, N.Y., 1986), pig (Hammer et al., Nature, 315:343-5, (1985)), sheep (Hammer et al., Nature, 315:343-5, (1985)), cattle, rat, or primate. See also, e.g., Church, Trends in Biotech., 5:13-19 (1987); Clark et al., Trends in Biotech., 5:20-4, (1987)); and DePamphilis et al., BioTechniques, 6:662-80 (1988)). Transgenic animals can be produced by the methods described in U.S. Pat. No. 5,994,618, and utilized for any of the utilities described therein.

Conditions Related to PDE2A

The present invention relates to the treatment, diagnosis, prevention, etc. in any disease, disorder, or condition that is related to, or mediated by, a PDE of the present invention, including mediated by any of the signaling pathways of which the PDEs are a member.

PDE2As of the instant invention are involved in a variety of functions and activities, and aberrant expression and/or activity of these phosphodiesterases can be associated with a variety of disease and conditions. This invention relates, e.g., to the detection (e.g., determination of the presence or absence) and/or quantitation of polypeptides or polynucleotides of the invention that are related to such conditions; and to the diagnosis and/or prevention, treatment, or amelioration of symptoms of such PDE2A-mediated or PDE2A-related conditions. The invention also relates to methods of identifying agents that modulate (i.e., increase or decrease) the expression and/or activity of polypeptides or polynucleotides associated with such conditions, and to methods of identifying polypeptide or polynucleotide alterations or mutants that are associated with such conditions. Furthermore, PDE2As of the invention are involved in the formation of memory, particularly long-term memory. Therefore, the invention also relates to agents and/or methods to stimulate the formation of memory in “normal” subjects (i.e., subjects who do not exhibit an abnormal or pathological decrease in a memory function), e.g., ageing middle-aged subjects.

Increased expression and/or activity of a PDE2A, with its concomitant decrease in the amount of intracellular cAMP or cGMP, is associated, e.g., with neurological conditions (e.g., memory impairment). In a particularly preferred embodiment, methods of the invention relate to conditions associated with brain-related (neurological) impairment, e.g., conditions associated with memory loss, especially long-term memory loss, or other dementias. In a further preferred embodiment, the methods of the invention relate to methods for enhancing memory in normal subjects.

In the brain, the level of cAMP or cGMP within neurons is believed to be related to the quality of memory, especially long term memory. Without wishing to be bound to any particular mechanism, it is proposed that since PDE2A degrades cAMP or cGMP, the level of this enzyme affects memory in animals, for example, in humans. For example, a compound that inhibits cAMP phosphodiesterase (PDE) can thereby increase intracellular levels of cAMP, which in turn activate a protein kinase that phosphorylates a transcription factor (cAMP response binding protein), which transcription factor then binds to a DNA promoter sequence to activate genes that are important in long term memory. The more active such genes are, the better is long-term memory. Thus, by inhibiting a phosphodiesterase, long term memory can be enhanced.

The condition of memory impairment is manifested by impairment of the ability to learn new information and/or the inability to recall previously learned information. Among the memory-related conditions that are affected by PDE2A levels and/or activity are, e.g., mild cognitive impairment (MCI) and age-related cognitive decline (e.g., cerebral senility). The present invention also relates to memory impairment as a result of disease (e.g., Huntington's disease), Down's syndrome, schizophrenia (e.g., paranoid, disorganized, catatonic, undifferentiated, or residual type), depression, or stroke. In another application, the invention relates to memory loss from chemical exposure (e.g., anesthetics or chemotherapy), radiation treatment, post-surgical trauma, or injuries (e.g., head trauma).

The present invention relates to dementias in general, which are diseases that include memory loss and additional intellectual impairment separate from memory. The invention relates to memory impairment in all forms of dementia. Dementias are classified according to their cause and include: neurodegenerative dementias (e.g., Alzheimer's Disease, Parkinson's Disease, Huntington's Disease, or Pick's Disease), vascular (e.g., infarcts, hemorrhage, cardiac disorders), mixed vascular and Alzheimer's Disease, bacterial meningitis, Creutzfeld-Jacob Disease, multiple sclerosis, traumatic (e.g., subdural hematoma or traumatic brain injury), infectious (HIV), genetic (e.g., Down's Syndrome), toxic (e.g., heavy metals, alcohol, or some medications), metabolic (e.g., vitamin B12 or folate deficiency), CNS hypoxia, Cushing's Disease, psychiatric (e.g., depression and schizophrenia), and hydrocephalus.

The invention also relates to states characterized by decreased NMDA function (e.g., schizophrenia), bipolar or manic depression, major depression, depression associated with psychiatric and neurological disorders, drug addiction (e.g., alcohol or morphine addiction), enhanced wakefulness, mood, movement, and anxiety disorders (e.g., panic disorder, agoraphobia, social phobia, obsessive-compulsive disorder, post-traumatic stress disorder, or acute or generalized anxiety disorder), psychosis (including psychosis induced by alcohol, amphetamines, cannabis, cocaine, hallucinogens, inhalants, opioids, or phencyclidine). Conditions that relate to the invention also include, e.g., stroke, multi-infarct dementia, amyolaterosclerosis (ALS), and multiple systems atrophy (MSA).

Increase in cAMP is also associated with increase in the health of neurons. Therefore, the invention also relates to the prevention of neurodegeneration resulting from disease or injury. Increase in cAMP is also known to relax smooth muscle and therefore, the invention is further related to the management of vasospasm associated with traumatic subarachnoid haemorrhage.

The present invention also relates to the diagnosis, treatment, and/or prophylaxis of any disease or condition mediated by a PDE2 of the present invention, including, but not limited to, any of the neurological conditions or disease mentioned above, inflammation, inflammatory diseases (e.g., asthma, rheumatoid arthritis, atopy, etc.), immune and immuno-modulatory disorders, neoplasia (includes both cancer and pre-cancerous lesions), heart disease (including, e.g., modulating heart rate), chronic obstructive pulmonary disease, adrenal gland disorders, ACTH (e.g., deficiency or excess) related conditions, Cushing disease, endocrine disorders, related to angiogenesis (e.g., to inhibit angiogenesis in conditions such as cancer diabetic retinopathy, neovascular glaucoma, psoriasis, retrolental fibroplasias, angiofibroma, or to promote angiogenesis in conditions such as ischemic syndromes and arterial obstructive disease), etc.

Screening for Modulatory Agents and Assays for PDE2A Levels and/or Activities

This invention provides methods of screening agents, in vitro or in vivo (e.g., in cell-based assays or in animal models), to identify those agents that modulate (e.g., enhance, stimulate, restore, inhibit, block, stabilize, destabilize, increase, facilitate, up-regulate, activate, amplify, augment, induce, decrease, down-regulate, diminish, lessen, reduce, etc.) synthesis and/or activity of PDE2As of the invention. Agents that inhibit such synthesis and/or activity (antagonists) may, e.g., result in an increased cyclic AMP or GMP levels within the subject cells and resultant physiological alterations resulting therefrom. Agents that enhance such synthesis and/or activity (agonists) may, e.g., result in a decreased cyclic AMP level within the subject cells. For example, antagonists can inhibit interaction of cAMP or cGMP with PDE2As of the invention disclosed herein, and agonists can enhance interactions of cAMP or cGMP with PDE2As. Such agents may, e.g., modulate phosphodiesterase activity, or inhibit or enhance cyclic nucleotide hydrolysis. The agents can also act indirectly, e.g., to diminish or enhance the levels of cytokines, such as TNF-alpha and beta, interferon-gamma, interleukins and chemokines that are involved e.g., in the response to inflammation.

Agents which inhibit PDE2A expression and/or activity (sometimes referred to herein as “PDE2A inhibitors”) can be used to treat, prevent, and/or ameliorate the symptoms of conditions associated with an over expression or increased activity of a PDE2A; and agents which enhance such activity can be used to treat, prevent, and/or ameliorate the symptoms of conditions associated with an underexpression or decreased activity of a PDE2A. Inhibitors of PDE2As can serve in general as anti-inflammatory and immunomodulatory agents. More specifically, they can be used, e.g., to treat any of the conditions described elsewhere herein which are associated with an overproduction of, or increased activity of, PDE2A. Stimulators of PDE2As can be used, e.g., to treat any of the conditions described elsewhere herein which are associated with an underproduction of, or decreased activity of, PDE2A.

Examples of agents which modulate PDE2As of the present invention, include but are not limited to, (Z)-5-fluoro-2-methyl-(4-pyridylidene)-3-(N-benzyl)indenylac-etamide hydrochloride (as well as other compounds disclosed in U.S. Pat. Pub. No. 20030232862); erythro-9-(2-hydroxy-3-nonyl)adenine (EHNA); sulindac sulfone and derivatives, CP248 and CP461 (Soh et al., Clin. Cancer Res., 6:4136-4141, 2000).

In assaying for potential antagonists or agonists, a variety of functions and/or enzymatic activities, which are associated with the full length PDE2As or with the novel 14-mer or 17-mer polypeptides thereof of the invention can be employed. Typical functions and activities are discussed elsewhere herein. Such assays can be performed using any suitable cell or tissue. In a preferred embodiment, assays are performed on cells or tissues in which PDE2As are highly expressed, e.g., placenta cells. In a most preferred embodiment, assays are performed on cells related to memory, such as, e.g., hippocampal tissue or cells. Assays can be performed in vitro, ex vivo or in vivo. In vivo assays can be performed using, e.g., transgenic or knock-out mice as already described, or a humanized mouse in which a human gene coding for the human isoform of PDE2A disclosed herein is present in place of the mouse gene otherwise coding for such analog. When agents that affect memory are being tested, they can be assayed directly in systems which measure components of memory, e.g., long-term memory. Methods for showing a correlation between cAMP and/or PDE2A levels and memory are routine in the art.

Methods to assay for the effects of putative inhibitors or stimulators of phosphodiesterases are conventional and well known in the art. For example, conventional assays are available to measure (e.g., quantitate) intracellular levels of cAMP or cGMP. In one embodiment, stable cell lines, such as CHO or QM7 cells that express a PDE2A of the invention, are treated with a putative modulatory agent, and the total level of intracellular cAMP or cGMP is measured. An increase in cAMP or cGMP levels indicates a PDE2A inhibitory activity by the agent being tested, while a decrease in cAMP or cGMP levels indicates an activating effect by the agent being tested.

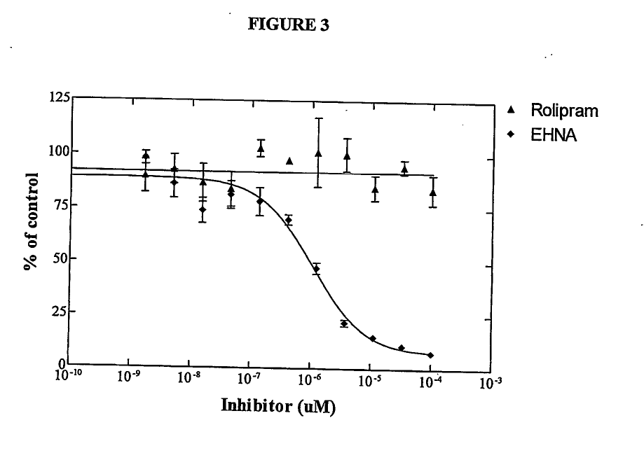

For example, FIG. 3 herein shows the effect of a known inhibitor, EHNA, on a PDE2A4 of the present invention (using recombinant PDE2A4 of the present invention expressed in a QM7 cell expression system).

Other conventional methods can be used to measure the binding affinity of putative inhibitors or stimulators of a phosphodiesterase, or to measure the ability of a putative inhibitor or enhancer to stimulate or inhibit interaction between the phosphodiesterase and a target molecule which normally interacts with it (e.g., a cyclic nucleotide or another component of the signal pathway with which the phosphodiesterase normally interacts (e.g., PKA or other components involved in cAMP turnover)). An example of an assay for an antagonist combines a PDE2A of the invention (i.e., a PDE2A4 isoform) and a potential antagonist (i.e., an inhibitor) under appropriate conditions for a competitive inhibition assay.

Other conventional methods to determine the levels of PDE2 polypeptides and polynucleotides, or to determine the presence of mutations therein, are well-known in the art. See, e.g., discussions below concerning diagnostic assays.

Any of the assays described herein can, of course, be adapted to any of a variety of high throughput methodologies, as can the generation, identification and characterization of putative inhibitory or stimulatory agents. Agents identified on the basis of their ability to modulate PDE2A expression or activity may also be used for modulating other PDEs, and/or for diagnosing or treating disease conditions related thereto.

Potential modulators, e.g., inhibitors or activators, of the invention, include, e.g., small chemical compounds (e.g., inorganic or organic molecules), polypeptides, peptides or peptide analogs, polynucleotides, antibodies that bind specifically to the polypeptides of the invention, or the like. Typical polypeptide agents include, e.g., mutant PDE2As or fragments thereof which exhibit impaired enzymatic activity but which have a higher affinity for a target than does wild type PDE2A; such polypeptides can out compete PDE2A and, thus, inhibit its activity. Other inhibitory or stimulatory substances may enter cells and bind directly to the DNA neighboring the sequences coding for the polypeptides of the invention, thereby decreasing their expression and thus increasing intracellular levels of cAMP or cGMP, or increasing their expression and thus decreasing intracellular levels of cAMP or cGMP.

One class of modulators includes small molecules that bind to and occupy the catalytic site of the polypeptide, thereby making the catalytic site inaccessible to a substrate such that normal biological activity is prevented. Catalytic sites can be determined by conventional, art-recognized methods, e.g., comparison to catalytic sites found in related phosphodiesterases. For example, phosphorodiesterases often include the catalytic signature sequence, HXXDHXX, wherein X is any amino acid. Examples of such small molecules include but are not limited to small chemical compounds, especially those having cyclic nucleotide-like structures.

Antisense Oligonucleotides and Ribozymes

Potential antagonists or inhibitors of the invention include isolated antisense oligonucleotides, or antisense constructs, which express antisense oligonucleotides, both of which classes of molecules can be prepared using conventional technology. Antisense technology can be used to control gene expression through methods based on binding of a polynucleotide to DNA or RNA. Without wishing to be bound to any particular mechanism, types of antisense oligonucleotides and proposed mechanisms by which they function include, e.g., the following: The 5′ coding portion of a polynucleotide sequence which encodes for a mature polypeptide of the present invention can be used to design an antisense oligonucleotide (e.g., an RNA, DNA, PNA etc. oligonucleotide) of any site which is compatible with the invention, e.g., of from about 10 to 40 base pairs in length. The antisense oligonucleotide can hybridize to the mRNA and block translation of the mRNA molecule into a PDE2A polypeptide (see e.g., Okano, J. Neurochem., 56:560 (1991); Oligodeoxynucleotides as Antisense Inhibitors of Gene Expression, CRC Press, Boca Raton, Fla. (1988)). Alternatively, an oligonucleotide can be designed to be complementary to a region of the gene involved in transcription (see, e.g. Lee et al., Nucl. Acids Res., 6:3073 (1979); Cooney et al., Science, 241:456 (1988); and Dervan et al., Science, 251:1360 (1991)), thereby preventing transcription and the production of PDE2A isoforms. For further guidance on administering and designing antisense, see, e.g., U.S. Pat. Nos. 6,200,960, 6,200,807, 6,197,584, 6,190,869, 6,190,661, 6,187,587, 6,168,950, 6,153,595, 6,150,162, 6,133,246, 6,117,847, 6,096,722, 6,087,343, 6,040,296, 6,005,095, 5,998,383, 5,994,230, 5,891,725, 5,885,970, and 5,840,708.

Antisense polynucleotides can comprise modified, nonnaturally-occurring nucleotides and linkages between the nucleotides (e.g., modification of the phosphate-sugar backbone; methyl phosphonate, phosphorothioate, or phosphorodithioate linkages; and 2′-O-methyl ribose sugar units), e.g., to enhance in vivo or in vitro stability, to confer nuclease resistance, to modulate uptake, to modulate cellular distribution and compartmentalization, etc. Any effective nucleotide or modification can be used, including those already mentioned, as known in the art, etc., e.g., disclosed in U.S. Pat. Nos. 6,133,438; 6,127,533; 6,124,445; 6,121,437; 5,218,103 (e.g., nucleoside thiophosphoramidites); and 4,973,679; Sproat et al., “2′-O-Methyloligoribonucleotides: synthesis and applications,” Oligonucleotides and Analogs A Practical Approach, Eckstein (ed.), IRL Press, Oxford, 49-86 (1991); Iribarren et al., “2′O-Alkyl Oligoribonucleotides as Antisense Probes,” Proc. Natl. Acad. Sci. USA, 87:7747-51 (1990); Cotton et al., “2′-O-methyl, 2′-O-ethyl oligoribonucleotides and phosphorothioate oligodeoxyribonucleotides as inhibitors of the in vitro U7 snRNP-dependent mRNA processing event,” Nucl. Acids Res., 19:2629-35 (1991). Effective amounts of antisense oligonucleotides as described above can be administered to a patient in need thereof by conventional means.

Antisense oligonucleotides can also be delivered to cells via, e.g., plasmids or other vectors, wherein the antisense sequence is operably linked to an expression control sequence. In this manner, RNA or DNA antisense is expressed in a cell and inhibits production of PDE2As, especially PDE2A4. A total length of about 36 nucleotides can be used in cell culture with cationic lipisomes to facilitate cellular uptake, but for in vivo use, preferably shorter oligonucleotides are administered, e.g., about 25 nucleotides.

In another embodiment, ribozymes corresponding to specific sequences, e.g., polynucleotides encoding sequences of the invention or fragments thereof, can be introduced into cells such that they cleave PDE2A4 coding or regulatory sequences. Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. Ribozyme molecules designed to catalytically cleave target gene mRNA transcripts can also be used to prevent translation of target gene mRNA and expression of target gene. (See, e.g., PCT International Publication WO90/11364, published Oct. 4, 1990; Sarver et al., Science, 247:1222-5 (1990)). While ribozymes that cleave mRNA at site-specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature, 334:585-91 (1988). For example, there are hundreds of potential hammerhead ribozyme cleavage sites within the nucleotide sequence of PDE2A4 sequences of the invention. Preferably the ribozyme is engineered so that the cleavage recognition site is located near the 5′ end of the target mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., Science, 224:574-8 (1984); Zaug and Cech, Science, 231:470-5 (1986); Zaug, et al., Nature, 324:429-33 (1986); published International patent application No. WO 88/04300 by University Patents Inc.; Been and Cech, Cell, 47:207-16 (1986)). The Cech-type ribozymes have an eight base pair active site, which hybridizes to a target RNA sequence whereafter cleavage of the target RNA takes place. The invention encompasses those Cech-type ribozymes which target eight base-pair active site sequences that are present in target gene.

As in the antisense approach, the ribozymes can be composed of modified oligonucleotides (e.g., for improved stability, targeting, etc.) and should be delivered to cells, which express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes, unlike antisense molecules, are catalytic, a lower intracellular concentration is required for efficiency.

Diagnostics/Assays for PDE2A

The present invention provides for a means of diagnosing or staging actual or potential disease conditions involving altered levels of cAMP or cGMP (e.g., which are mediated by or related to phosphodiesterase production or activity) by determining the amounts (e.g., the presence or absence, or the quantity) of the polypeptides of the invention, or their levels of activity, in an animal suspected of having such a disease condition or being at risk therefor. For example, the invention provides a process for diagnosing a disease in an animal afflicted therewith, or diagnosing a susceptibility to a disease in an animal at risk thereof, wherein said disease is related, for example, to an over- or under-expression or activity of a phosphodiesterase according to the present invention, comprising determining the amount of said phosphodiesterase or the level of said phosphodiesterase activity in a cell from said animal, wherein said animal is preferably a mammal and most preferably a human.

When assaying samples for diagnostic purposes, using any of the methods described herein, samples may be obtained from any suitable cell, tissue, organ, or bodily fluid from a patient, including but not limited to blood, urine, saliva, tissue biopsy and autopsy material. In one embodiment, samples for diagnosis are taken from cells or tissues in which high or moderate levels of PDE2A4 expression are normally observed, e.g., placenta, kidney, pancreas, skeletal muscle or neurological tissue. In a preferred embodiment, the disease conditions to be diagnosed involve loss of memory as a primary or secondary effect thereof, especially loss of long term memory, and the cells tested are typically neurons, especially those of the brain, for example, neurons of the hippocampal region (e.g., in hippocampal slices).

Enzymatic assays for the various activities exhibited by PDE2As are conventional. Some such assays are described above and in the examples. Detection and/or quantitation of protein levels can be accomplished by any of a variety of conventional methods, e.g., methods based on antibodies or antigen-specific fragments of the invention. Immunological assays include, e.g., ELISA, RIA and FACS assays. A two-site, monoclonal-based immunoassay, utilizing antibodies reactive to two non-interfering epitopes on a PDE2A polypeptide are preferred, but a competitive binding assay may be employed. These and other assays are described, e.g., in Hampton et al., Serological Methods, a Laboratory Manual, APS Press, St. Paul, Minn. (1990).

The invention provides methods for diagnosing a disease or susceptibility thereto wherein said disease is related to production of an aberrant form of a phosphodiesterase according to the invention, e.g., one resulting from a genetic mutation. Such aberrant (or variant) proteins include those described above, e.g., proteins having amino acid substitutions, deletions, inversions, insertions, rearrangements (e.g., as a result of aberrant splicing events) or inappropriate post-translational modifications. Aberrant proteins may exhibit increased or decreased activity of any of the functions described elsewhere herein. Aberrant proteins may also exhibit increased or decreased interactions with other proteins, such as, e.g., protein kinases, cytoskeletal proteins, etc. Aberrant expression and/or activity can occur as a result of a mutation directly in the polypeptide, or in upstream or downstream regulatory regions, such as in 5′ upstream, promoter and/or enhancer regions, in introns, etc. Such mutations can be nucleotide deletions, additions, substitutions, SNPs, etc.

Variant can be detected by any of a variety of conventional methods. For example, antibodies or antigen binding fragments can be used to detect the presence of aberrant forms of the polypeptides disclosed herein, using immunological methods such as those described above.

In accordance with the present invention, an antibody or antigen-binding fragment can be present in a kit, where the kit includes, e.g., one or more antibodies or antigen-binding fragments, a desired buffer, detection compositions, proteins (e.g., wild type) to be used as controls, etc.

Assays involving polynucleotides can be used to determine the presence or absence of a nucleic acid in a sample and/or to quantify it, or to detect a mutation or polymorphism. Such assays can be used, e.g., for diagnostic, prognostic, research, or forensic purposes. The assays can be, e.g., membrane-based, solution-based, or chip-based. Assays can be performed at the single-cell level, or in a sample comprising many cells, where the assay is “averaging” expression over the entire collection of cells and tissue present in the sample.

Any suitable assay format can be used, including, but not limited to, Southern blot analysis, Northern blot analysis, polymerase chain reaction (“PCR”) (e.g., Saiki et al., Science, 241:53 (1988); U.S. Pat. Nos. 4,683,195, 4,683,202, and 6,040,166; PCR Protocols: A Guide to Methods and Applications, Innis et al., eds., Academic Press, New York, 1990), reverse transcriptase polymerase chain reaction (“RT-PCR”), anchored PCR, rapid amplification of cDNA ends (“RACE”) (e.g., Schaefer in Gene Cloning and Analysis: Current Innovations, pp. 99-115 (1997)), ligase chain reaction (“LCR”) (EP 320 308), one-sided PCR (Ohara et al., Proc. Natl. Acad. Sci. USA, 86:5673-7 (1989)), indexing methods (e.g., U.S. Pat. No. 5,508,169), in situ hybridization, differential display (e.g., Liang et al., Nucl. Acid. Res., 21:3269-75 (1993); U.S. Pat. Nos. 5,262,311, 5,599,672 and 5,965,409; WO97/18454; Prashar and Weissman, Proc. Natl. Acad. Sci. USA, 93:659-63, and U.S. Pat. Nos. 6,010,850 and 5,712,126; Welsh et al., Nucleic Acid Res., 20:4965-70 (1992), and U.S. Pat. No. 5,487,985) and other RNA fingerprinting techniques, nucleic acid sequence based amplification (“NASBA”) and other transcription based amplification systems (e.g., U.S. Pat. Nos. 5,409,818 and 5,554,527; WO 88/10315), polynucleotide arrays (e.g., U.S. Pat. Nos. 5,143,854, 5,424,186; 5,700,637, 5,874,219, and 6,054,270; PCT WO 92/10092; PCT WO 90/15070), QBeta Replicase (PCT/US87/00880), Strand Displacement Amplification (“SDA”), Repair Chain Reaction (“RCR”), nuclease protection assays, subtraction-based methods, Rapid-Scan™, etc. Additional useful methods include, but are not limited to, e.g., template-based amplification methods, competitive PCR (e.g., U.S. Pat. No. 5,747,251), redox-based assays (e.g., U.S. Pat. No. 5,871,918), Taqman-based assays (e.g., Holland et al., Proc. Natl. Acad, Sci. USA, 88:7276-80 (1991); U.S. Pat. Nos. 5,210,015 and 5,994,063), real-time fluorescence-based monitoring (e.g., U.S. Pat. No. 5,928,907), molecular energy transfer labels (e.g., U.S. Pat. Nos. 5,348,853, 5,532,129, 5,565,322, 6,030,787, and 6,117,635; Tyagi and Kramer, Nature Biotech., 14:303-9 (1996)). Any method suitable for single cell analysis of gene or protein expression can be used, including in situ hybridization, immunocytochemistry, MACS, FACS, flow cytometry, etc. For single cell assays, expression products can be measured using antibodies, PCR, or other types of nucleic acid amplification (e.g., Brady et al., Methods Mol. & Cell. Biol., 2:17-25 (1990); Eberwine et al., Proc. Natl. Acad. Sci. USA, 89:3010-14 (1992); U.S. Pat. No. 5,723,290). These and other methods can be carried out conventionally, e.g., as described in the mentioned publications.

The invention provides methods for diagnosing a disease in an animal afflicted therewith, or diagnosing susceptibility to a disease in an animal at risk thereof, wherein said disease is related, for example, to an over- or under-expression of a polynucleotide encoding a phosphodiesterase according to the invention, comprising determining the amount of said polynucleotide in a cell from said animal, wherein said animal is preferably a mammal and most preferably a human. Any of the assay methods described herein, or otherwise known in the art, can be used to determine the presence of and/or to quantitate, such polynucleotides.

Furthermore, detection of a mutated or polymorphic form of a gene allows a diagnosis of a disease or a susceptibility to a disease which results from expression of a mutated PDE2A polypeptide that may have, for example, increased or decreased activity in degrading cAMP or cGMP. Such mutations include, e.g., any of those described elsewhere herein, e.g., point mutations, insertions, deletions, substitutions, transversions, and chromosomal translocations.

Individuals carrying mutations in a gene of the present invention may be detected at the DNA level by a variety of techniques. Genomic DNA may be used directly for detection or may be amplified enzymatically by using PCR (Saiki et al., Nature, 324:163-6 (1986); Innis et al. eds., PCR Protocols: A Guide to Methods in Amplification, Academic Press, New York (1996)) prior to analysis. RNA or cDNA may also be used for the same purpose. As an example, PCR primers complementary to the nucleic acid encoding the novel 14-mer of PDE2A4 can be used to identify and analyze mutations. For example, deletions and insertions can be detected by a change in size of the amplified product in comparison to the normal genotype. Point mutations can be identified, e.g., by hybridizing amplified DNA to radiolabeled RNA or radiolabeled antisense DNA sequences. Perfectly matched sequences can be distinguished from mismatched duplexes by a variety of methods, including, e.g., RNase A digestion or by differences in melting temperatures. Rapid sequencing methods can be employed.

Sequence differences between the reference gene and genes having mutations may be revealed by the direct DNA sequencing method. In addition, cloned DNA segments may be employed as probes to detect specific DNA segments. The sensitivity of this method is greatly enhanced when combined with PCR. For example, a sequencing primer is used with double-stranded PCR product or a single-stranded template molecule generated by a modified PCR. The sequence determination is performed by conventional procedures with radiolabeled nucleotide or by automatic sequencing procedures with fluorescent-tags.

A polynucleotide sequence coding for part or all of a novel 14-mer or 17-mer of the invention may act as a reference for the development of probes, e.g., as long as 30 to 45 nucleotides, or longer, that can be used to probe the genome of animals suspected of being at risk for disease, or having such disease. Probes corresponding to regulatory sequences e.g., sequences which govern the amount of mRNA coding for the PDE2A4s of the invention, or of the PDE2A protein produced, can also be used. Such regulatory sequences include, e.g., promoter or enhancer elements, sequences, which govern splicing events, stability of nucleic acid or protein, termination/polyadenylation and/or intracellular localization of mRNAs or proteins.

Genetic testing based on DNA sequence differences may be achieved by detection of alteration in electrophoretic mobility of DNA fragments in gels with or without denaturing agents. Small sequence deletions and insertions can be visualized by high-resolution gel electrophoresis. DNA fragments of different sequences may be distinguished on denaturing formamide gradient gels in which the mobilities of different DNA fragments are retarded in the gel at different positions according to their specific melting or partial melting temperatures (see, e.g., Myers et al., Science, 230:1242 (1985)), or by mass spectroscopy analysis.

In addition, sequence changes at specific locations may also be revealed by nuclease protection assays, such as RNase and S1 protection or the chemical cleavage method (e.g., Cotton et al., Proc. Natl. Acad. Sci. USA, 85:4397-4401 (1985)) and these are deemed within the methods of the invention.

Thus, the detection of a specific DNA sequence may be achieved by methods such as, e.g., hybridization, RNase protection, chemical cleavage, direct DNA sequencing or the use of restriction enzymes, (e.g., Restriction Fragment Length Polymorphisms (RFLP)) and Southern blotting of genomic DNA.

In addition to more conventional gel-electrophoresis and DNA sequencing, mutations can also be detected by in situ analysis.

Mutations in regulatory elements (e.g., promoter, enhancer, introns, etc.) can also affect the level of polynucleotide (e.g., mRNA) or protein made, and can give rise to disease symptoms. Such mutations include, e.g., mutations in promoter or enhancer elements, splice signals, termination and/or polyadenylation signals; mutations which result in truncated proteins, such as chain terminators; sites involved in feed-back regulation of nucleic acid or polypeptide production, etc. Diagnostic methods to detect such mutations in regulatory elements are conventional.

In accordance with the present invention, a polynucleotide can be present in a kit, where the kit includes, e.g., one or more polynucleotides, a desired buffer (e.g. phosphate, tris, etc.), detection compositions, RNA or cDNA from different tissues to be used as controls, libraries, etc. The polynucleotide can be labeled or unlabeled, with radioactive or non-radioactive labels as known in the art. Kits can comprise one or more pairs of polynucleotides for amplifying nucleic acids specific for a PDE2A, e.g., comprising a forward and reverse primer effective in PCR. These include both sense and anti-sense orientations. For instance, in PCR-based methods (such as RT-PCR), a pair of primers are typically used, one having a sense sequence and the other having an antisense sequence.

Other Uses of Polynucleotides

The sequences of the present invention are also valuable for chromosome identification. The polynucleotides coding for the sequences of the invention, and homologs thereof, are specifically targeted to and can hybridize with a particular location on an individual human chromosome. Moreover, there is a current need for identifying particular sites on the chromosome, for example, as part of the human genome project. Thus, sequences can be mapped to chromosomes, e.g., by preparing PCR primers (preferably 15-25 bp) from the cDNA.

Fluorescence in situ hybridization (FISH) of a cDNA clone to a metaphase chromosomal spread can likewise be used to provide a precise chromosomal location in one step. This technique can be used with cDNA having at least 50 or 60 bases. For a review of this technique, see Verma et al., Human Chromosomes: A Manual of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis (coinheritance of physically adjacent genes).

One can determine the differences in the cDNA or genomic sequence between affected and unaffected individuals. If a mutation is observed in some or all of the affected individuals but not in any normal individuals, then the mutation is likely to be a causative agent of the disease. With current resolution of physical mapping and genetic mapping techniques, a cDNA precisely localized to a chromosomal region associated with the disease could be one of between 50 and 500 potential causative genes. (This assumes 1 megabase mapping resolution and one gene per 20 kb).

A fragment of a polynucleotide of the present invention may also be used as a hybridization probe, e.g., for a cDNA or genomic library to isolate a full length cDNA (or genomic DNA) and to isolate other cDNAs (or genomic DNAs) which have a high sequence similarity to the gene or similar biological activity. Probes of this type preferably have at least 7 or 8 bases, more preferably about 10, 11, 12, 13, 14 or 15 bases, and most preferably at least about 30 bases, and exhibit about 65-100% sequence identity to part or all of the sequence coding for the novel 14-mer and 17-mer disclosed in SEQ ID NOS 4 and 16 of FIGS. 2 and 7. Such probes may also have 45 or more bases but again contain sequences which exhibit about 65-100% sequence identity to a sequence coding for some or all of a novel 14-mer or 17-mer polypeptides of the invention, or a variant thereof. Because of the degeneracy of the genetic code, many sequences exist which exhibit a high degree of sequence identity to sequences coding for part or all of a novel 14-mer or 17-mer disclosed herein. The set of such sequences also includes those that code for amino acid sequences that are themselves homologous to these sequences. Hybridization probes are specific to, or for, a selected polynucleotide. The phrases “specific for” or “specific to” a polynucleotide have a functional meaning that the probe can be used to identify the presence of one or more target genes in a sample. The probe is specific in the sense that it can be used to detect a polynucleotide above background noise (“non-specific binding”).

Therapeutics

The methods of the present invention are also directed to facilitating the development of potentially useful therapeutic agents that may be effective in combating PDE2A mediated or related disease conditions, and to methods of effecting such treatments. The invention also provides methods to enhance or restore memory function in “normal” subjects, e.g., by activating brain, especially hippocampal, neuronal cAMP or cGMP phosphodiesterase, particularly the PDE2A activity disclosed herein, and thereby decreasing levels of cAMP or cGMP in such cells.

Any agent which modulates the expression and/or activity of PDE2A polypeptide or polynucleotide of the invention, e.g., a PDE2A modulating agent identified by an art recognized assay, such as those herein, can be used therapeutically. Some such agents are discussed elsewhere herein.

Agents which affect expression and/or activities of polypeptides of the invention can be administered to patients in need thereof by conventional procedures, in order to prevent or treat disease conditions as disclosed elsewhere herein and/or to ameliorate symptoms of those conditions. Such agents can be formulated into pharmaceutical compositions comprising pharmaceutically acceptable excipients, carriers, etc., using conventional methodologies. Formulations and excipients, which enhance transfer (promote penetration) of an agent across the blood-brain barrier are also well known in the art.

In addition to agents, which can moderate the expression or activity of a phosphodiesterase, treatment methods according to the invention also encompass the administration of a phosphodiesterase (e.g., a PDE2A4 or PDE2A2) or variant or fragment thereof to a patient in need of such therapy. For example, such a polypeptide or fragment can compensate for reduced or aberrant expression or activity of the protein, and/or, by virtue of, e.g., higher affinity for a target, can provide effective competition for it. In another embodiment, conventional methods of immunotherapy can be used.

Polynucleotides of the invention can also be used in methods of gene therapy, e.g., utilized in gene delivery vehicles. The gene delivery vehicle may be of viral or non-viral origin. See generally, Jolly, Cancer Gene Therapy, 1:51-64 (1994); Kimura, Human Gene Therapy, 5:845-52 (1994); Connelly, Human Gene Therapy, 1:185-93 (1995); and Kaplitt, Nature Genetics, 6:148-153 (1994). Gene therapy vehicles for delivery of constructs including a coding sequence of a therapeutic of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses, which are constructed to carry or express a selected nucleic acid molecule of interest. Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, Cancer Res., 53:3860-4 (1993); Vile and Hart, Cancer Res., 53:962-7 (1993); Ram et al., Cancer Res., 53:83-8 (1993); Takamiya et al., J. Neurosci. Res., 33:493-503 (1992); Baba et al., J. Neurosurg., 79:729-35 (1993); U.S. Pat. No. 4,777,127; GB Patent No. 2,200,651; and EP 0 345 242. Preferred recombinant retroviruses include those described in WO 91/02805.

Packaging cell lines suitable for use with the above-described retroviral vector constructs may be readily prepared (see PCT publications WO 95/30763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs alpha virus-based vectors that can function as gene delivery vehicles. Such vectors can be constructed from a wide variety of alpha viruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250 ATCC VR-1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir., 63:3822-8 (1989); Mendelson et al., Virol, 166:154-65 (1988); and Flotte et al., Proc. Natl. Acad. Sci. USA, 90:10613-17 (1993).

Representative examples of adenoviral vectors include those described by Berkner, Biotechniques, 6:616-27 (Biotechniques); Rosenfeld et al., Science, 252:431-4 (1991); WO 93/19191; Kolls et al., Proc. Natl, Acad. Sci. USA, 215-19 (1994); Kass-Eisler et al., Proc. Natl. Acad. Sci. USA, 90:11498-502 (1993); Guzman et al., Circulation, 88:2838-48 (1993); Guzman et al., Cir. Res., 73:1202-7 (1993); Zabner et al., Cell, 75:207-16 (1993); Li et al., Hum. Gene Ther., 4:403-9 (1993); Cailaud et al., Eur. J: Neurosci., 5:1287-91 (1993); Vincent et al., Nat. Genet., 5:130-4 (1993); Jaffe et al., Nat. Genet., 1:372-8 (1992); and Levrero et al., Gene, 101:195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to killed adenovirus as described in Curiel, Hum. Gene Ther., 3:147-54 (1992), may be employed.

Other gene delivery vehicles and methods may be employed, including polycationic condensed DNA linked or unlinked to killed adenovirus alone, for example, Curiel, Hum. Gene Ther., 3:147-54 (1992); ligand-linked DNA, for example, see Wu, J. Biol. Chem., 264:16985-7 (1989); eukaryotic cell delivery vehicles cells, for example see U.S. Ser. No. 08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell Biol., 14:2411-18 (1994) and in Woffendin, Proc. Natl. Acad. Sci. USA, 91:1581-5 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13796, WO 94/23697 and WO 91/14445, and EP No. 0 524 968.

Further non-viral delivery suitable for use includes mechanical delivery systems such as the approach described in Woffendin et al., Proc. Natl. Acad. Sci. USA, 91(24):11581-5 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

Computer-Based Applications

The nucleotide or amino acid sequences of the invention are also provided in a variety of media to facilitate use thereof. As used herein, “provided” refers to a manufacture, other than an isolated nucleic acid or amino acid molecule, which contains a nucleotide or amino acid sequence of the present invention. Such a manufacture provides the nucleotide or amino acid sequences, or a subset thereof (e.g., a subset of open reading frames (ORFs)) in a form which allows a skilled artisan to examine the manufacture using means not directly applicable to examining the nucleotide or amino acid sequences, or a subset thereof, as they exist in nature or in purified form.

In one application of this embodiment, a nucleotide or amino acid sequence of the present invention can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a nucleotide or amino acid sequence of the present invention.

As used herein, “recorded” refers to a process for storing information on computer readable medium. The skilled artisan can readily adopt any of the presently known methods for recording information on computer readable medium to generate manufactures comprising the nucleotide or amino acid sequence information of the present invention.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of dataprocessor structuring formats (e.g., text file or database) in order to obtain computer readable medium having recorded thereon the nucleotide sequence information of the present invention.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. Search means are used to identify fragments or regions of the sequences of the invention, which match a particular target sequence or target motif.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen on a three-dimensional configuration, which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software include, but are not limited to, MacPattern (EMBL), BLASTN and BLASTX (NCBIA).

For example, software, which implements the BLAST (Altschul et al., J. Mol. Biol., 215:403-10 (1990)) and BLAZE (Brutlag et al., Comp. Chem., 17:203-7 (1993)) search algorithms on a Sybase system can be used to identify open reading frames (ORFs) of the sequences of the invention which contain homology to ORFs or proteins from other libraries. Such ORFs are protein encoding fragments and are useful in producing commercially important proteins such as enzymes used in various reactions and in the production of commercially useful metabolites.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius; and, unless otherwise indicated, all parts and percentages are by weight.

### EXAMPLES

### Example 1

Identification and Cloning of N-Terminal Human PDE2A4

Two primers, hsPDE2A-5′R and hsPDE2A-5′N, were designed according to the exon sequences common to all known PDE2A isoforms. To discover 5′ alternatively spliced isoforms, hsPDE2A-5′R was used together with the RACE primer (Ambion) to PCR the human brain RACE-Ready cDNA library (Ambion). The reaction was carried out using PCRx system and platinum HF polymerase (Invitrogen) with the following cycling characteristics: 94° C. for 3′ for 1 cycle; 94° C. for 30″ and 68° C. for 1′30″ for 35 cycles; 68° C. for 7′ for 1 cycle. The resulting PCR products were then used as templates for the nested PCR with hsPDE2A-5′N and NEST primer (Ambion) following the same condition as RACE PCR reaction. The resulting fragments were cloned into pcDNA3.1v5/his TOPO vector and sequenced. One clone, hsPDE2A-5′N#15, contained the N-terminal of a novel isoform.

| (SEQ ID NO:5) | |
|---|---|
| hsPDE2A-5′R: 5′-CAGTTTCCACTCGGGGGAGCACAGCTGAC | |
| (SEQ ID NO:6) | |
| hsPDE2A-5′N: 5′-CCTGAAATGTCGATGACAGAGCCCAGACTCAGC | |
| hsPDE2A-5′N#15: | |
| (SEQ ID NO:7) | |
| CGGGAGCAGCGGGGCAGCAGAGCTGGATTGGGGTGTTGAGTCCAGGCTGAGTAGGGGGCAGCCCACTGCTCTTGGTCCCT | 80 |
| GTGCCTGCTGGGGGTGCCCTGCCCTGAACTCCAGGCAGCGGGGACAGGGCGAGGTGCCACCTTAGTCTGGCTGGGRAGGC | 160 |
| GGACGATGGGGAGTGATGGGGCAGGCATGCGGCCACTCCATCCTCTGCAGGAGCCAGCAGTACCCGGCAGCGCGACCGGC | 240 |
| TGAGCCGATGATTCTGAAGAATCTGGAAGTGCAGAGCTTAGCCCCTGGCATGCGGCAGGTGCTCACAAAGAAGTTTACAG | 320 |
| CTTCCTGAGCACTGTTTCCACACCTGTGATCTCATTTAATCCTCACCACAAACCCAAGAGACTGCTGTTTTCCGGATGAA | 400 |
| GAAACAGAGGATCCAGGAGGGGAAATCGCTTGCCCACAGGCGGGGCCAGCAGGTCTTCCTCAAGCCGGACGAGCCGCCGC | 480 |
| CGCCGCCGCAGCCATGCGCCGACAGCCTGCAGGACGCCTTGCTGAGTCTGGGCTCTGTCATCGACATTTCAGG | 553 |

### Example II

Full-Length Cloning of a Novel Isoform of Human PDE2A, hsPDE2A4

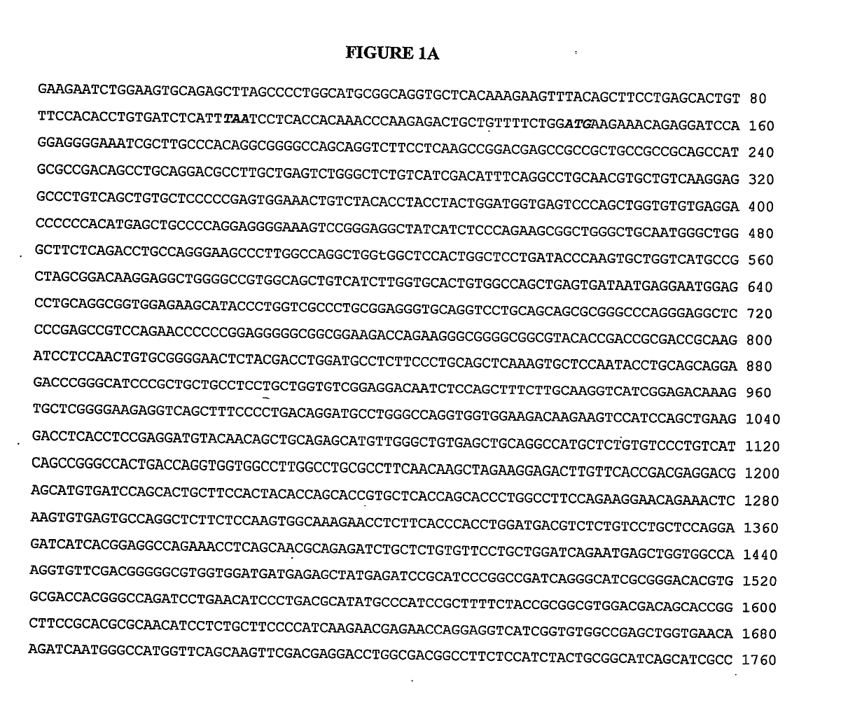

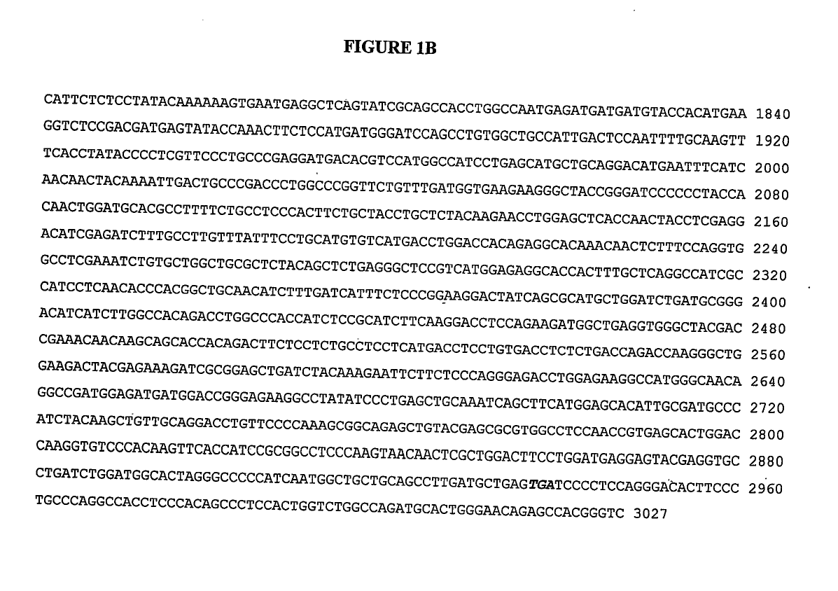

A hsPDE2A4 specific primer, hsPDE2A4-5′a, and a hsPDE2A 3′ primer, hsPDE2A-3′a, were designed and used to PCR human hippocampus cDNA. The reaction was carried out using PCRx system and platinum HF polymerase (Invitrogen) with the following cycling characteristics: 94° C. for 5′ for 1 cycle; 94° C. for 30″, 65° C. for 30″ and 68° C. for 3′ for 35 cycles; 68° C. for 7′ for 1 cycle. The resulting fragment (˜3.0 kb) was cloned into pcDNA3.1v5/his TOPO vector and sequenced. The cDNA and amino acid sequences for hsPDE2A4 are shown in FIGS. 1 and 2.

(SEQ ID NO:8)

hs2A4-5′a: 5′-GAAGAATCTGGAAGTGCAGAGCTTAGCCCCTGG

(SEQ ID NO:9)

hs2A-3′a: 5′-ACCCGTGGCTCTGTTCCCAGTGCATCTG

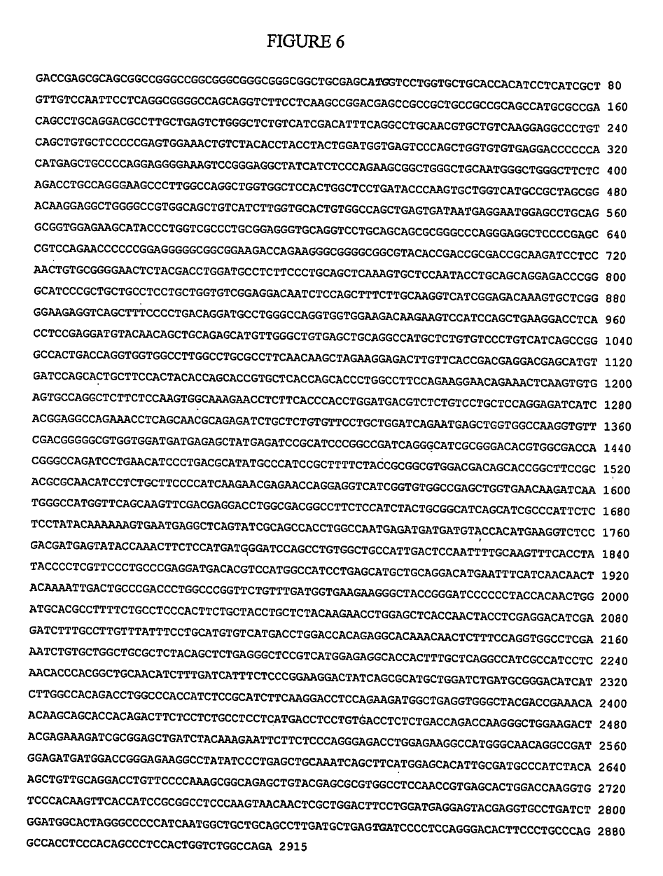

In the same 5′ RACE reaction as described in Example I, sequence that is highly homologous to the unique N-terminal sequence of rat PDE2A2 was detected. This sequence indicates the existence of human PDE2A2. The human ortholog can be isolated using standard cloning methods. For example, primers can be designed and used to PCR appropriate human libraries to isolate human orthologs. The human PDE2A2 sequences are shown in FIGS. 6 and 7.

### Example III

Human PDE2A4 Enzyme Properties

To express in active form, human PDE2A4 cDNA was cloned downstream of the CMV promoter into pcDNA3.1/V5-His-TOPO vector, and the plasmid was transiently transfected into QM-7 cells by lipofectamine-plus reagent (Invitrogen). Forty-eight hours after the transfection, the QM-7 cells were lysed in buffer containing 50 mM Tris/HCl, 10 mM NaCl, 5 mM EGTA, 1 mM EDTA, 1 mM DTT and proteinase inhibitor cocktail (Roche). The cell lysate was used as a source for recombinant hsPDE2A4 enzyme.

The enzyme activity assay was performed according to Thompson et al, Ann N Y Acad Sci, 185:36-41 (1971) and Alvarez et al, Anal Biochem, 203:76-82 (1992) in the presence of 50 mM Tris/HCl, pH 8.0, 5 mM MgCl2, 4 mM 2-mercaptoethanol and 0.3 mg/ml BSA. The activity of human PDE2A4 was analyzed for both cAMP and cGMP. The experiments were performed in duplicates for each substrate concentration. The Km and Vmax were calculated with Michaelis-Menten equation on Prism software.

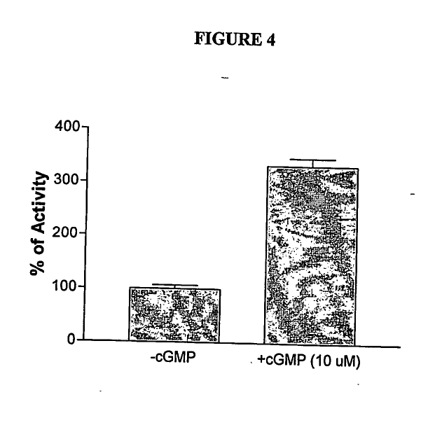

QM-7 cell lysate containing recombinant human PDE2A4 hydrolyzes both cAMP and cGMP (Table 1). Human PDE2A4 exhibits higher affinity for cGMP (˜2 fold higher) than for cAMP (Francis et al., Progress in Nucleic Acid Research and Molecular Biology, 65:1-52 (2001)). The maximum speed of hydrolysis, however, is about 5 times faster for cAMP (Table 1). Other reports have showed similar Vₘₐₓ for cAMP and cGMP (Rosman G J, et al., Gene 191:89-95 (1997); Francis et al., Progress in Nucleic Acid Research and Molecular Biology, 65:1-52 (2001)). It is unlikely that endogenous cGMP from the QM-7 cell lysate causes the elevated Vₘₐₓ for cAMP, since exogenous cGMP still shows stimulated effect (FIG. 4).

TABLE 1
Enzymatic properties of human PDE2A4

| cAMP | cGMP |
|---|---|
| Kₘ = 39.6 ± 5.3 μM | Km = 17.5 ± 2.2 μM |
| Vₘₐₓ = 1649.0 ± 72.3 pmol/mg/min | Vₘₐₓ = 334.4 ± 16.0 pmol/mg/min |

The enzymatic activity of recombinant human PDE2A4 is inhibited by EHNA, a PDE2 specific inhibitor (IC₅₀=1.1 μM). Rolipram, a specific inhibitor for PDE4, has no inhibitory effect on human PDE2A4 enzyme activity (FIG. 3).

The enzymatic activity of recombinant human PDE2A4 can be stimulated by cGMP, a unique characteristic for PDE2 gene family (FIG. 4). The activity for hydrolyzing cAMP was elevated more than three-fold in the presence of 10 μM cGMP (FIG. 4). The increase in enzymatic activity is most likely due to increase in affinity for cAMP resulted from cGMP binding to the GAF domain.

### Example IV

Tissue Distribution of Human PDE2A Isoforms

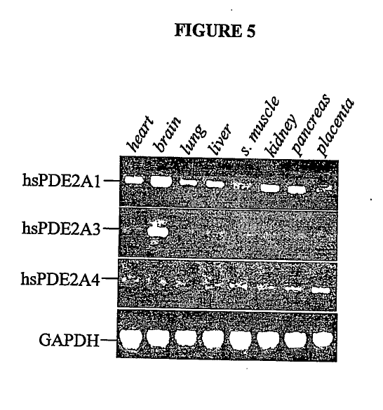

The following primers were used to analyze tissue distribution pattern of human PDE2A isoforms by semi-quantitative PCR: hsPDE2A1 (hs2A1-5′ and hs2A-3′c); hsPDE2A3 (hs2A-5′d and hs2A-3′c); hsPDE2A4 (hs2A4-5′a and hs2A-3′c). The primer combinations were used to PCR human MTC panels with PCRx system and platinum HF polymerase (Invitrogen). The PCR reactions were allowed to proceed for 35 cycles. The results of the PCR were analyzed on 2% agarose gels (FIG. 5).

(SEQ ID NO:10)

hs2A1-5′: 5′-GACGACCGACTGGAGGACGCCTTGCTGAG

(SEQ ID NO:11)

hs2A-5′d: 5′-CACCCCACCTTAGTCTGGCTGGGGAGGCGGAC

(SEQ ID NO:12)

hs2A-3′c: 5′-GCCACAGTGCACCAAGATGACAGCTGCCAC

All human PDE2A isoforms tested were widely expressed in all the tissues on the panel. The relative level of expression, however, was greatly different among tissues and isoforms. PDE2A3 is very prominent in brain and only weakly detectable in all other tissues. PDE2A4 is expressed the highest in placenta. PDE2A1 expression is readily detectable in several tissues, with the strongest expression in brain, yet only weakly expressed in skeletal muscle and placenta. The differential expression pattern suggests non-redundant functional roles of human PDE2A isoforms in various tissues. Different size bands were also detected in various tissues for each PDE2A isoform, e.g., PDE2A3 in the brain (FIG. 5). These bands could represent undiscovered isoforms.

The topic headings set forth above are meant as guidance as to where certain information can be found in the application. They are not intended to be the only source in the application where information on such a topic can be found.

From the foregoing description, one skilled in the art can easily ascertain the essential characteristics of this invention, and without departing from the spirit and scope thereof, can make changes and modifications of the invention to adapt it to various usage and conditions.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of all applications, patents and publications, cited above and in the figures are hereby incorporated in their entirety by reference, including U.S. Provisional Application Ser. No. 60/459,977, filed Apr. 4, 2003.

1. An isolated polynucleotide comprising a polynucleotide sequence coding for a human phosphodiesterase 2A2, or a mutation thereof.

2. An isolated polynucleotide comprising a polynucleotide sequence coding for a mammalian phosphodiesterase 2A4, or a mutation thereof.

3. An isolated polynucleotide for a phosphodiesterase 2A2, comprising:
(a) a polynucleotide sequence set forth in FIG. 6 (SEQ ID NO: 13),
(b) a polynucleotide sequence coding for a polypeptide having the amino acid sequence set forth in FIG. 7 (SEQ ID NO: 14),
(c) a polynucleotide sequence which hybridizes under stringent conditions to the polynucleotide sequence set forth in FIG. 6 (SEQ ID NO: 13), wherein said conditions comprise washing in 0.1×SSC and 0.1% SDS for 30 min at 65° C.,
(d) a polynucleotide sequence coding for a polypeptide sequence comprising amino acids 1-17 of the amino acid sequence set forth in FIG. 7 (SEQ ID NO: 16), or
specific fragments thereof, or complements thereto.

4. An isolated polynucleotide for a phosphodiesterase 2A4, comprising:
(a) a polynucleotide sequence set forth in FIG. 1 (SEQ ID NO: 1),
(b) a polynucleotide sequence coding for a polypeptide having the amino acid sequence set forth in FIG. 2 (SEQ ID NO: 2),
(c) a polynucleotide sequence which hybridizes under stringent conditions to the polynucleotide sequence set forth in FIG. 1 (SEQ ID NO: 1), wherein said conditons comprise washing in 0.1×SSC and 0.1% SDS for 30 min at 65° C.,
(d) a polynucleotide sequence coding for a polypeptide sequence comprising amino acids 1-14 of the amino acid sequence set forth in FIG. 2 (SEQ ID NO: 4),
(e) the complete polynucleotide sequence coding for a phosphodiesterase 2A4 of the cDNA clone contained in the plasmid deposited with the ATCC as Deposit Designation No.
PTA-4986, or
specific fragments thereof, or complements thereto.

5. An isolated polynucleotide of any of claims 1-4, which codes without interruption for said phosphodiesterase 2A2 or 2A4.

6. An isolated polynucleotide of claims 3 or 4, wherein said hybridization conditions further comprise hybridizing in 5×SSC, 0.5% SDS, 100 pg/ml denatured salmon sperm DNA, and 50% formamide, at 42° C.

7. An isolated polynucleotide of claims 3 or 4, and which comprises a polynucleotide sequence coding for its N-terminal polypeptide sequence, said polypeptide having at least 80% sequence identity along its entire length to SEQ ID NO: 4 or 16.

8. An isolated polynucleotide of any of claims 1-4, wherein said polynucleotide codes for a polypeptide having phosphodiesterase activity.

9. An expression vector, comprising a polynucleotide of any of claims 1-4 operably linked to a promoter sequence.

10. A transfected host cell, comprising a polynucleotide of any of claims 1-4.

11. A transfected host cell of claim 10, wherein said polynucleotide codes for a polypeptide having phosphodiesterase activity.

12. An isolated polypeptide which is coded for a polynucleotide sequence of any of claims 1-4.

13. A method for identifying an agent that modulates the expression or activity of a phosphodiesterase in transfected host cells, comprising: contacting a transfected host cell of any of claim 10 with a test agent under conditions effective for said test agent to modulate the expression or activity of said phosphodiesterase, and determining whether said test agent modulates the expression or activity of said phosphodiesterase.

14. A method of claim 13, wherein said determining comprises measuring amounts of cAMP or cGMP produced in the presence of said agent.

15. A method of claim 14, wherein the agent inhibits the expression or activity of said phosphodiesterase.

16. An antibody which is specific for a polypeptide of claim 12.

17. A non-human transgenic mammal comprising a polynucleotide of any of claims 1-4 coding for a phosphodiesterase.

18. A mammalian cell whose genome comprises a functional disruption of an endogenous phosphodiesterase encoding a polynucleotide of any of claims 1-4.

19. A non-human transgenic animal comprising a cell of claim 18.

20. A non-human transgenic animal of claim 17, which has impaired cognitive function.

21. A method of selecting a polynucleotide sequence or polypeptide coding for a phosphodiesterase from a database comprising polynucleotide or polypeptide sequences, comprising, displaying, in a computer-readable medium, a polynucleotide sequence of any of claims 1-4, or a polypeptide encoded by said polynucleotide sequence, wherein said displayed sequences have been retrieved from said database upon selection by a user.

22. A non-human transgenic animal of claim 19, which has impaired cognitive function.
